(19) United States
(12) Patent Application Publication
(10) Pub. No.: US 2004/0151774 A1
(43) Pub. Date: Aug. 5, 2004

(54) Therapeutic compositions for drug delivery to and through covering epithelia

(76) Inventors: Giovanni M. Pauletti, Loveland, OH (US); Kishorkumar J. Desai, Westchester, OH (US); Susan L. Roweton, Raleigh, NC (US); Donald C. Harrison, Cincinnati, OH (US); Lynda M. Sanders, Los Altos, CA (US)

Correspondence Address: HANA VERNY, PETERS, VERNY, JONES & SCHMITT, L.L.P., SUITE 230, 425 SHERMAN AVENUE, PALO ALTO, CA, 94306, US

(21) Appl. No.: 10/698,794

(22) Filed: Oct. 31, 2003

Related U.S. Application Data
(63) Continuation-in-part of application No. 10/444,634, filed on May 22, 2003.
(60) Provisional application No. 60/425,655, filed on Nov. 12, 2002.
(60) Provisional application No. 60/423,260, filed on Oct. 31, 2002.
(60) Provisional application No. 60/424,920, filed on Nov. 8, 2002.

(51) Int. Cl. A61K 9/14

(52) U.S. Cl. 424/486; 424/488

(57) ABSTRACT

Polymer foams and films for delivery of therapeutic agents to and through nasal, oral or vaginal mucosa and cornified or non-cornified epithelium of labia and scrotum. Polymer foams or absorbable or non-absorbable films containing a therapeutic agent incorporated therein wherein said agent is released from said foams or films upon placement of said foam or film on the surface epithelium of nasal, oral, or vaginal labia or scrotum. The foam or the film has a controllable rate of gelling, swelling and degradation and is preformed into a device or is applied as a coating to a surface of a more complex drug delivery system.

This application is a continuation-in-part of the patent application Ser. No. 10/444,634 filed on May 22, 2003 and is based on and claims priority of the Provisional applications Ser. No. 60/425,655 filed on Nov. 12, 2002, Ser. No. 60/423,260 filed on Oct. 31, 2002, Ser No. 60/424,920 filed on Nov. 8, 2002.

### BACKGROUND OF THE INVENTION

1. Field of the Invention

The present invention concerns therapeutic compositions suitable for delivery of therapeutic agents to and through covering epithelia of nasal, oral or vaginal cavities as well as through the epithelium of labia and scrotum. In particular, the invention concerns the compositions comprising a therapeutic agent and a polymer, further optionally in combination with mucoadhesive agents, penetrations enhancers, release modifiers and/or other additives and excipients. These compositions may be prepared as biodegradable or non-biodegradable foams or films of solid structure or semi-solid or liquid preparation comprising a therapeutic agent incorporated therein wherein said agent is released from said compositions upon placement thereof on the surface of or in the close proximity of a nasal, buccal, vaginal, labial or scrotal epithelium. Depending on a presence of specific components present in said compositions, the compositions of the invention act either locally on the covering epithelium or are delivered through such epithelium to a systemic circulation. The compositions of the invention have a controllable rate of gelling, swelling and degradation. The compositions are either preformed into a device such as a foam tampon, tampon-like cylinder, strip, pad, pillow, tube, sheet, sphere, tablet, ring or bead or single or double sided film sheet or are applied, as one component, to a surface of a more complex drug delivery system which comprises, as a second component, a device made of a different material, such as a conventional tampon, tampon-like device, pessary, ring, strip, pad, pillow, sheet, tube, sphere, tablet or a bead covered by said composition. Liquid composition is supplied and stored as a sprayable system which upon spraying onto an epithelial surface rapidly gels into a foam layer. The film is either preformed into sheets of a desirable shape and size or is sprayed onto the mucosal, labial or scrotal epithelial surface wherein it gels and forms the foam, film or gel or is applied to a surface and covers and coats such surface of the vaginal, nasal, buccal, scrotal or labial device.

2. Background of the Invention and Related Disclosures

The skin, scrotal and labial epithelium and mucous membranes such as those that line the vagina or nasal and oral cavity, serve as a protective barrier against the outside environment so that bacteria and viruses are excluded and prevented from entering the body through this route. Besides excluding harmful bacteria and viruses, the above described barrier is also very effective at excluding chemicals, drugs and pharmacological agents that are applied to the skin, labia, scrotum or mucosa. This barrier is composed of several layers.

In the skin, the stratum corneum represents a cornified layer, epidermis is formed of a layer of stratified squamous epithelial cells, dermis is formed of a thin layer of cells that interdigitates with the epidermis and a basement membrane covers the capillary plexus leading to the systemic circulation.

Like the skin, the covering epithelium of nasal, vaginal or oral cavities, labia and scrotum are lined by multiple layers of stratified, squamous epithelium that forms a protective barrier for exclusion of bacteria and other foreign substances. The epithelium lining the nasal, vaginal or oral cavity represents the surface of a mucus-secreting mucosa. Mucosa is thus a mucus-secreting membrane lining body cavities and canals. Labia is formed by non-mucosal non-cornified epithelium. Scrotum is formed by non-mucosal lightly cornified epithelium which is not the same as the cornified layer of the skin.

Because of the presence of the barrier preventing the entry of bacteria, viruses and various chemicals, problems were encountered with attempted delivery of pharmacological agents through these tissues. Consequently, the therapeutic effect of nasal, buccal or vaginal medications were, until now, confined primarily to the external or internal topical use. It would thus be advantageous to provide compositions which would conveniently, efficiently and practically permit a drug delivery topically or to the systemic circulation via nasal, buccal, vaginal, labial or scrotal epithelium.

In order to permit passage of pharmacological agents through the skin barrier, attempts were made to discover and/or develop compounds which would enhance their penetration through these barriers. The most well known of these penetration enhancers is dimethyl sulfoxide (DMSO). DMSO has the ability to rapidly alter the cell membrane characteristics to allow substances to pass between the cells, into the cell and through the cell. These unique characteristics have made this compound useful in the laboratory as a permeation enhancer and as a cryoprotectant for cell freezing. Unfortunately DMSO is not safe for human use and has been banned for human use by the Food and Drug Administration.

A second skin permeation enhancer, ethoxydiglycol, known under its trade name TRANSCUTOL®, has been recently developed and introduced for topical use and is primarily used to promote delivery of skin tanning agents into the epidermis and into the dermal layer of the skin.

In vitro evaluation of ethoxydiglycol as permeation enhancer for transdermal delivery of clonazepam is described in Eur. J. Pharm. Sci., 9:365-372 (2000). This publication evaluates the influence of ethoxydiglycol alone or in combination with propylene glycol, on clonazepam permeation through an artificial membrane and on excised (ex vivo) rabbit ear skin from carbopol hydrogels. The article describes an increase of drug permeation through the skin as a function of ethoxydiglycol content in the formulation, and concludes that ethoxydiglycol is a good enhancing carrier for clonazepam and increases the flux of the drug into the skin and across the skin if combined with propylene glycol which has penetration and carrier properties.

Until recently, however, ethoxydiglycol has not been used for or shown to promote the transmucosal delivery of the drug across the nasal, buccal and vaginal mucosa or through the labia or scrotum into the systemic circulation or described to have such properties. Prior use of ethoxydiglycol to promote transvaginal delivery was disclosed by inventors and such use is described in patents U.S. Pat. Nos. 6,086,909, 6,197,327 B1, 6,416,779 B1, 6,572,874 B1 and pending application Ser. Nos.: 10/226,667 filed on Aug. 21, 2002 and 10/349,029 filed on Jan. 22, 2003, all hereby incorporated by reference.

While these patents and applications describe mucosal and transmucosal drug delivery, they do not describe in great details such delivery using a biodegradable or non-degradable compositions, although these compositions could provide advantage of being efficacious, convenient, practical, simple, functional, soft and pliable and non-intrusive when prepared and easily confirming to a surface of the scrotal, labial, vaginal, oral or nasal epithelium when sprayable or dried into a film when prepared as foams and films and easily confirming to a surface of cornified and non-cornified epithelia.

Thus, it would be advantageous to have available therapeutic compositions which would promote delivery of pharmaceutical agents to the cornified or non-cornified epithelium of the labia, scrotum, vaginal, nasal or oral cavity and facilitate access of these pharmacologically active agents locally or through these tissues into the general systemic circulation.

Transvaginal compositions for delivery of drugs to the uterus through vaginal mucosa have been recently discovered and described in U.S. Pat. Nos. 6,086,909, 6,416,779 B1, 6,572,874 B1 and 6,197,327 B1. These compositions are typically prepared as transmucosal formulations or, preferably, as a device incorporated with said transmucosal formulation.

It has now been discovered that specifically formulated compositions, particularly those formulated into solid, semi-solid or liquid foams or films can overcome generally observed problems caused by the above described protective barriers which effectively prevent translabial, transscrotal or transmucosal drug delivery through the nasal, buccal, vaginal, labial or scrotal epithelium into the general circulation.

It is therefore an object of the present invention to provide a therapeutically useful compositions for delivery of therapeutic agents to and through cornified and non-cornified epithelia lining the nasal, oral, or vaginal cavity and the labia and scrotum. Such delivery comprises compositions formed into biodegradable or non-degradable foam and film formulations that are soft, pliable, and non-intrusive when prepared and easily conformable to the surface of the scrotum, labia, nasal, oral, or vaginal cavity.

All patents, patent applications and publications cited herein are hereby incorporated by reference.

### SUMMARY OF THE INVENTION

One aspect of the present invention is a therapeutically useful composition comprising at least a substrate polymer compound or a mixture thereof and a therapeutically effective agent formulated into a biodegradable or non-degradable foam or film of different rigidity and viscosity as solid, semi-solid, or liquid formulation.

Another aspect of the current invention is a therapeutic composition comprising a substrate polymer formulated into a biodegradable or non-degradable solid, semi-solid or liquid foam or film, said composition additionally containing a mucoadhesive agent, release modifier, penetration enhancer, sorption promoter and/or another pharmaceutically acceptable excipient and additive.

Still another aspect of the current invention is a polymeric foam or film composition particularly suitable for a vaginal, nasal, buccal, labial, scrotal topical or transepithelial delivery of therapeutically effective agents locally topically or to the general circulation.

Yet another aspect of the current invention is a polymeric foam or film composition having incorporated therein a therapeutically effective agent selected from the group consisting of anti-inflammatory agents, local anesthetics, calcium channel antagonists, potassium channel blockers, β-adrenergic agonists, vasodilators, cyclooxygenase inhibitors, antimicrobial, antiviral, antifungal, antipsychotic, anti-osteoporotic, anti-migraine, anti-HIV, anti-epileptic, anti-neoplastic, chemotherapeutic, anti-psychotic, anti-neurogenerative agents, opioid analgesics and biotechnology-derived pharmacological agents, such as proteins and peptides.

Still another aspect of the current invention is a method for using a polymeric bio-degradable or non-degradable foam or film compositions for delivery of therapeutic agents locally or systemically to the general blood circulation wherein said compositions comprise a therapeutically effective agent selected from the group consisting of anti-inflammatory agents, local anesthetics, calcium channel antagonists, potassium channel blockers, β-adrenergic agonists, vasodilators, cyclooxygenase inhibitors, antimicrobial, antiviral, antifungal, antipsychotic, anti-osteoporotic, anti-epileptic, anti-psychotic and-neurogenerative anti-migraine, anti-HIV, anti-neoplastic and chemotherapeutic agents and biotechnology-derived pharmacological agents, such as proteins and peptides.

Still yet another aspect of the current invention is a biodegradable or non-degradable mucosal, transmucosal, labial, translabial, scrotal and transscrotal foam or film composition for delivery of a therapeutic agent to and/or through nasal, buccal, vaginal, labial or scrotal epithelium, said composition consisting of from about 1 to about 95% of a polymer selected from the group consisting of microcrystalline cellulose, polyacrylic acid, polyethylene glycol, polypropylene glycol, divinyl glycol, polyethylene oxide, polypropylene oxide, carboxymethyl cellulose, hydroxyethyl cellulose, polylactide, polyglycolide, polymethacrylic acid, poly-γ-benzyl-L-glutamate, polypropylene fumarate, poly-ε-caprolactone, polybutylene terephthalate, polyvinyl alcohol, polyvinyl ether, poly-1-vinyl-2-pyrrolidinone, 2,5-dimethyl-1,5-hexadiene, divinyl benzene, polystyrene-divinyl benzene, polyanhydrides such as poly-bis(p-carboxy-phenoxypropane)-co-sebacic acid, polyhydroxyalkanoates, poly-β-hydroxybutyrate, poly-β-butyrolactone, alkyl-substituted silica gel, tetraethylorthosilicate, dimethyldiethoxysilane, pectin, collagen, or a mixture thereof, wherein said composition is prepared into a foam preformed into a device such as a tampon, tampon-like cylinder, strip, pad, pillow, tube, film, sheet, sphere, tablet, ring or bead, or prepared as a film, or incorporated into or applied, as one component, to a surface of a more complex drug delivery system which comprises, as a second component, a device made of different material, such as a conventional tampon, tampon-like device, pessary, ring, strip, pad, pillow, sheet, tube, sphere, tablet or a bead partially or totally covered or coated by said foam or film wherein said composition is supplied and stored as solid, semi-solid, or liquid preparation, which upon contact with the epithelial tissue or on the surface of a device maintains or rapidly changes the physical appearance to accommodate the anatomical and therapeutic needs at the site of administration.

Still yet another aspect of the current invention is a foam tablet or a dissolvable foam tablet for administration of a pharmacologically effective agent alone or incorporated into a device for insertion into nasal, oral or vaginal cavity or placed in close contact to the labia or scrotum.

Yet another aspect of the current invention is a biodegradable or non-degradable film comprising a pharmacologically effective agent suitable for placement on a surface of nasal, oral, vaginal, labial or scrotal epithelium.

### DEFINITIONS

As used herein:

“Covering epithelia” means tissues in which cells are organized in layers that cover the external surface or line cavities of the body. Histologically, epithelial tissues can be divided into covering epithelia and glandular epithelia. This invention concerns covering mucus-secreting epithelia, such as the nasal, buccal and vaginal but also covering labial and scrotal keratinized epithelia.

“Mucosal” means delivery of the drug locally to the vaginal, nasal or buccal mucus-secreting epithelia.

“Transmucosal” means delivery of the drug systemically through the vaginal, nasal or buccal mucus-secreting epithelia into the systemic circulation.

“Buccal” means delivery of the pharmaceutical agent to the mucosa lining the oral cavity.

“Labial” means delivery of the pharmaceutical agent locally to the labia.

“Translabial” means delivery of the pharmaceutical agent systemically through the non-mucosal non-cornified labial epithelium to the systemic circulation.

“Scrotal” means delivery of the drug locally to the scrotum.

“Transscrotal” means delivery of the drug systemically through the scrotal non-mucosal lightly cornified epithelium into the systemic circulation.

“Cornified” means keratinized tissue.

“Agent”, “pharmacologically effective agent”, “pharmacologically acceptable agent”, “pharmacological agent”, “an active pharmacologically acceptable agent” or “drug” means a natural or synthetic chemical compound which induces a biological or therapeutic effect when administered to a mammal, including human subject, through the mucosal or labial or scrotal epithelium.

“Pharmaceutical agent” or “pharmaceutically acceptable agent” means an excipient, typically pharmacologically inactive.

“Release modifier” or “carrier” means a compound able to aid in the release of the drug from the composition.

“Alginic acid” means alginic acid or a salt thereof, such as alginic acid sodium salt.

“Non-ionizable glycol derivative” means a synthetic or non-naturally occurring conjugate of aliphatic glycol or a conjugate of aliphatic glycol with aliphatic or aromatic alcohol or ether, such as ethoxydiglycol known under its trade name TRANSCUTOL®, or mixtures thereof.

“TRANSCUTOL®” means ethoxydiglycol also known under the name of diethyleneglycol monoethyl ether.

“AVICEL®” means microcrystalline cellulose of nominal size 50 microns, commercially available from FMC Biopolymers.

“NOVEON®” means polycarbophil or polyacrylic acid crosslinked by di-vinyl glycol.

“Poloxamer” means a family of ethylene oxide-propylene oxide block copolymers, also known as a copolymers of polyoxyethylene and polyoxypropylene.

“Carbopol” means polyacrylic acid polymers lightly cross-linked with a polyalkenyl polyether, commercially available from B. F. Goodrich.

### BRIEF DESCRIPTION OF FIGURES

FIG. 1 illustrates a release of ketorolac tromethamine from the alginic acid hydroxypropyl methylcellulose foams into a pH 4.2 phosphate buffer.

FIG. 2 shows a release of ketorolac from alginic acid film into a synthetic vaginal fluid at pH 4.2.

FIG. 3 shows a water uptake and dissolution of hydroxypropyl methylcellulose and hydroxypropyl methylcellulose-Avicel foams at different percentile mixtures.

### DETAILED DESCRIPTION OF THE INVENTION

The current invention describes therapeutically useful biodegradable or non-degradable foam or film compositions and a method for topical epithelial or transepithelial delivery of therapeutic agents to and across a nasal, buccal, vaginal, labial or scrotal epithelium into the general systemic circulation.

The foam or film compositions of the invention permit efficacious delivery of pharmacologically active agents locally directly to the vaginal, nasal or buccal epithelia or through a penetration of the vaginal, nasal, buccal, labial or scrotal epithelium into the general systemic circulation. The new compositions, in combination with new delivery routes, avoid problems connected with the oral administration which often leads to drug deactivation, or with the invasive intravenous, intramuscular, intraperitoneal, intracutaneous, cutaneous or subcutaneous routes of delivery requiring injections, visit to the doctor's office and/or assistance of medical personnel.

The newly discovered routes of topical epithelial or transepithelial nasal, buccal, vaginal, labial or scrotal administration are noninvasive, require no assistance by medical personnel or visit to the doctor's office, eliminate the need for excessive doses of the drug needed for oral delivery, and are altogether more convenient, practical and economical. The transepithelial delivery of drugs across the vaginal, nasal, buccal, labial, or scrotal epithelium according to the invention bypasses the gastrointestinal tract absorption, liver metabolism and kidney deactivation and delivers the drug locally or directly to the systemic blood circulation. Moreover, all foam or film compositions are eminently practical, non-intrusive and comfortable as they are soft and pliable and easily conformable to a tissue surface.

The foam compositions may be preformed into a structural foam which is either biodegradable or non-degradable and easily takes on the contouring of the tissue surface. The film compositions may be conveniently used alone as a one or multilayered one-sided or a two sided nasal, buccal, vaginal or labial film inserts or placed or sprayed on the scrotal and other tissue surface or used as a coating on the non-film devices, even as a coating on the foam device.

Moreover, the compositions of the invention, due to the chemical properties of their components combined with their processing, promote and permit delivery of the drug with variable chemical properties, such as drugs with variable drug stability, solubility and absorption into the tissue, and permit elimination of side effects observed with administration of higher doses of these drugs, because the drug is delivered locally or directly to the blood circulation aided by the composition's mucoadhesive, adhering and penetration properties. These variable chemical properties depend on the presence of a compound acting as a mucoadhesive or release modifying agent, typically a hydrophilic or hydrophobic polymer, alone or in a combination with another polymer, and/or further in combination with appropriate penetration enhancers or sorption promoters and/or release modifiers, depending on the drug.

I. Therapeutic Compositions

Therapeutic compositions according to the invention comprise essentially a hydrophilic or hydrophobic polymer component, preferably the hydrophilic polymer, in combination with a pharmacologically effective agent, said combination processed into a polymer foam or film. This combination has been found to efficaciously deliver the therapeutic agents to and through nasal, oral or vaginal mucosal epithelium as well as through the non-cornified or lightly cornified epithelium of labia and scrotum. A therapeutic agent incorporated into the foam or film is released from said composition upon placement of said composition on the surface of vaginal, nasal or oral mucosal epithelium and the epithelium of the labia and scrotum and acts either locally or penetrates through the tissue, or both. The foam or film of the invention has a controllable rate of gelling, swelling and degradation.

The foam or film composition of the invention comprises at least two components, namely a polymer, preferably a hydrophilic polymer or a mixture thereof, which typically has mucoadhesive or carrier properties, and a therapeutic agent or a mixture thereof, but may, additionally, contain another mucoadhesive agent, release modifier, penetration enhancer, sorption promoter and/or another pharmaceutically acceptable excipient and additive.

The foam or film compositions of the invention are particularly suitable for a topical and transepithelial vaginal, nasal, buccal, labial and scrotal delivery of therapeutic agents locally or to the general circulation. Representative therapeutic agents are anti-inflammatory agents, local anesthetics, calcium channel antagonists, potassium channel blockers, β-adrenergic agonists, vasodilators, cyclooxygenase inhibitors, antimicrobial, antiviral, antifungal, antipsychotic, anti-osteoporotic, anti-migraine, anti-HIV, anti-neoplastic, anti-epileptic, anti-neurodegenerative and chemotherapeutic agents, and biotechnology-derived pharmaceutical agents, such as proteins and peptides.

The compositions of the invention are preferably formulated into the solid, semi-solid or liquid foams or films.

A. Foam Formulations

The foam formulations suitable for delivery of pharmaceutical agents comprise a foam preformed into a specific shape of solid structure or a semi-solid or liquid preparation, which forms a foam layer upon contact with the epithelial tissue or the surface of a device. The pharmacologically effective agent may be incorporated before foam formation or by coating of the inner pores of a prefabricated polymeric foam scaffold or coating or surface of the foam or film.

Drugs and other additives can be added to a prefabricated polymeric foam scaffold by spraying the foam with a dilute solution of the drug or additive in methylene chloride or ethanol. Preferably the quantity of solution, the temperature, and the ambient air velocity are such that the solvent evaporates immediately after the solution is absorbed within the foam or on its surface. This process is similar to that used when applying coatings to pills.

The volume of solution applied per gram of foam is selected such that a substantial portion of the foam is coated. Having determined the appropriate solution volume, the drug concentration is selected so that the desired drug dose per unit weight or per unit volume is obtained.

Alternatively, drugs and additives can be incorporated by emulsion coating where water-in-oil or oil-in-water emulsions prepared in polymer solution is forced through a prefabricated foam scaffold by applying vacuum. After solvent evaporation, a polymer film containing the drugs and additives is then deposited on the porous scaffold surface. Processing parameters of this emulsion coating are known to the skilled in the art and any type of process, additives and equipment required to optimize stability and release of pharmacological agents from within the scaffold structure are intended to be within the scope of this invention.

1. Fabrication of Foams

The present invention concerns foam compositions suitable for delivery of therapeutic agents to and through the nasal, buccal, vaginal, labial, and scrotal cornified and non-cornified epithelia. Said compositions of biodegradable or non-degradable foams having solid, semi-solid, or liquid structure may be prepared by processes known in the art that introduce porosity in a polymer matrix, namely by lyophilization, aeration, freeze drying, hydrocarbon templating, salt or particulate leaching, gel or solvent casting, gas expansion, sintering, polymerization of high internal phase emulsions, and free form fabrication techniques such as three-dimensional polymer printing. The most preferred process to fabricate foams is lyophilization, which is described in detail below. Examples of the process applications that may be used to fabricate foams included in the invention have been disclosed previously. See, for example, Proc. Natl. Acad. Sci. USA, 97, 1970-1975 (2000); Polymer, 35, 1068-1077 (1994); J. Biomat. Sci. Polym. Ed., 7, 23-28 (1995); Biomaterials, 17, 1417-1422 (1996); J. Biomed. Mat. Res., 30, 449-461 (1996); J. Controlled Rel., 40, 77-87 (1996); Biomaterials, 24, 3133-3137 (2003) and J. Controlled Rel., 87, 57-68 (2003)).

Lyphilized foams are open cell, high-surface-area, biodegradable or non-degradable constructs that can be manufactured from a variety of polymers, preferably from hydrophilic polymers. The foam materials are characterized by controlled chemical and physical properties that can be tailored according to their intended application.

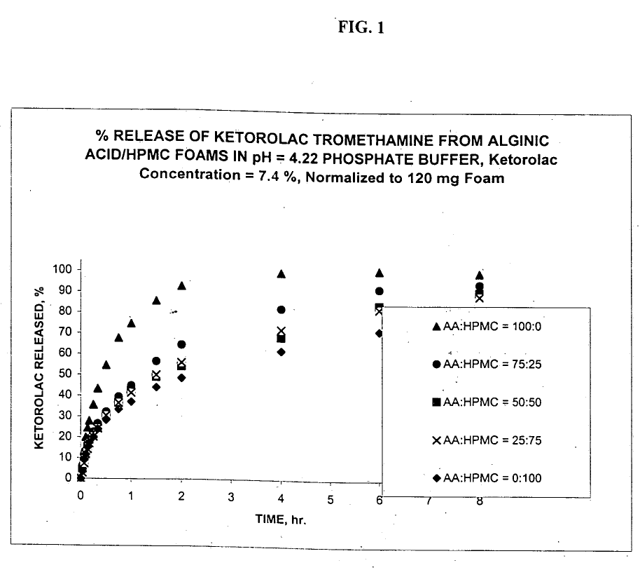

Tuneable properties include hydrophilicity, rate of fluid absorption, degradation profile and dissolution rate, a measure of which is the time needed to complete disappearance of the foam. The release of the drug, water uptake and dissolution of the foams or films are illustrated in FIGS. 1-3.

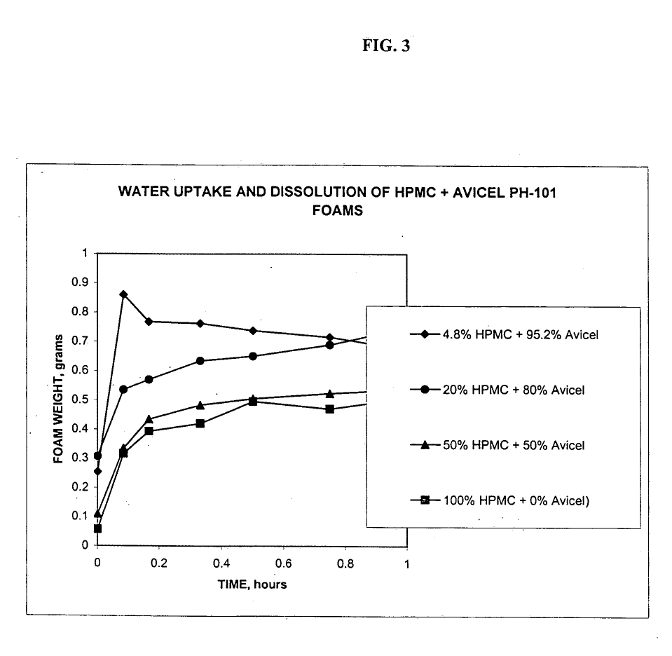

The invention thus can be a foam that hydrates and forms a gel quickly and is capable of dispersing over a relatively large area. The invention can also be a foam that hydrates and forms a gel slowly to provide sustained release of a therapeutic agent over hours or days. These properties are advantageously modifiable by changing polymers, ratios of the polymers to each other or to the drug and/or additives, as seen in FIGS. 1 and 3.

Typically, the lyophilized foam is prepared by dissolving an appropriate polymer, preferably a hydrophilic polymer, or a mixture thereof serving as a substrate material, as listed below in section C, in an amount needed to prepare solution from 1 to 10% (w/w) in an aqueous or non-aqueous solvent, such as methanol, ethanol, glycerine, methylene, chloride, propylene glycol, propylene carbonate, glycofurol, cetyl alcohol, difluroethane and isopropyl alcohol, preferably a purified water. Alternatively, polymeric solutions with the drug and additives may be prepared in acetic acid, cyclohexane, acetonitrile, tert-butanol, ethanol, and isopropanol or in mixtures of aqueous and non-aqueous solvents.

Compositions are prepared by dissolving an appropriate amount from about 0.01 to about 2000 mg or more, of a selected pharmacological agent or a mixture of two or more of such agents in a suitable solvent, preferably purified water, mixing this solution together with the polymer solution for from about 10 minutes to about several hours, preferably about 15-60 minutes, freezing said mixture at from −60° C. to about −100° C., preferably at −80° C., into a desirable shape, for example by pouring said mixture, before freezing, into a vial, pan, plate, tube, etc., of a desirable shape or into a foam sheet and, when frozen, cutting said sheet into a structure of a desirable shape and lyophilizing said frozen mixture by using any type of appropriate lyophilizer or lyophilizing equipment. Lyophilization conditions and apparatuses and equipment are known in the art and any type of lyophilization process or equipment is intended to be within the scope of this invention.

Typically, the polymer or polymer mixture and drug solution, as described above, is first frozen for at least 15 minutes, and typically at least 30 minutes, in a form having the shape and size desired for the finished lyophilized foam. For water solutions, the freezing temperature is from 0° C. to −80° C. and preferably less than −10° C. After freezing, the frozen samples are ejected or removed from the forms, optionally by brief warming on the outside of the forms. The frozen samples are placed in trays pre-cooled to a temperature below the freezing point of the solvent. While under vacuum, the samples are then converted to foams by lyophilization (freeze-drying) at 0° C. to −80° C. and preferably below −20° C. for about 48 hours to about 144 hours. Less time or more time may be required depending on the foam or film thickness and composition. After the water has been removed, the foams or films are warmed to room temperature, typically while still under vacuum. The procedure yields therapeutically useful foams or films containing a drug incorporated therein.

In the alternative, a closed-cell form can be prepared by aeration process. In this process, a polymer solution is rapidly mixed in a mixer such as Oakes mixer, by high-shear mixing blades, while air or another gas is injected. The resulting foam can be metered into molds or spread as a thin layer onto a substrate film. The foam can then be dried under ambient conditions or with heat.

Alternatively, the above foam can be frozen and lyophilized according to the procedures described above.

2. Biodegradable and Non-Degradable Foam

In one embodiment, this invention concerns compositions formulated into a foam for delivery of therapeutic agents to or through nasal, buccal, vaginal, labial, and scrotal epithelia. Physical and chemical properties of foams of the invention can be tailored to optimize their intended use, which is achieved by controlling the rate of release of the pharmacologically active agents incorporated into foams with said compositions. Drug release from the delivery device can occur by diffusion or erosion, or by a combination of both, leading to immediate, controlled, or pulsed delivery of the agent to or through the nasal, buccal, vaginal, labial, or scrotal epithelia.

The rate of drug release depends on physicochemical properties of the drug, the composition of the foam, and the surrounding media at the site of administration wherein pH, ionic strength, temperature, buffer capacity, enzyme activity, and cellular activity are only a few examples of variable that have an influence.

Foam scaffolds, fabricated from compositions that undergo degradation at the site of administration into smaller units or polymers by various mechanisms, are classified as biodegradable systems. Biodegradable polymers are preferably designed to allow drug release by bulk or surface erosion and include natural and synthetic polymers alone or in combination with representative but not limiting examples of polysaccharides such as alginate, dextran, cellulose, collagen, and chemical derivatives thereof, proteins such as albumin and gelatin and copolymers and blends thereof, polyhydroxy acids such as polylactides, polyglycolides and co-polymers thereof, polyethylene terephthalate, polybutiric acid, polyvaleric acid, polylactide-co-caprolactone, polyanhydrides, polyorthoesters, and blends and co-polymers thereof.

Non-degradable foam systems in this invention are the system wherein compositions resist a destruction of the three-dimensional function of the delivery system at the site of administration allowing drug release predominantly by diffusion from the composition. Representative but not limiting examples of non-biodegradable polymers that may be used exclusively or in combination with biodegradable polymers to fabricate foam compositions with desired characteristics as described by this invention include polyamides, polyethylene, polypropylene, polystyrene, polyvinyl chloride, polymethacrylic acid, and derivatives thereof alone or as co-polymeric mixtures thereof.

3. Shape of Foam

Foam compositions can be prepared by lyophilization in a range of sizes and a variety of shapes including foam films, sheets, pillows, tubes, cylinders, spheres, tablets, rings, beads or any other desirable shape using an appropriate processes known in the art that introduce porosity in a polymer matrix, namely lyophilization, aeration or freeze drying, hydrocarbon templating, salt or particulate leaching, gel or solvent casting, gas expansion, sintering, polymerization of high internal phase emulsions, and free form fabrication techniques such as three-dimensional polymer printing.

The foam is preformed into a device such as a tampon, tampon-like cylinder, strip, pad, pillow, tube, film, sheet, sphere, tablet, ring, bead or any other shape as might be desirable or is applied, as a one component, to a surface of a more complex drug delivery system which comprises, as a second component, a device made of a different material, such as, for example, a conventional vaginal tampon, tampon-like device, pessary, ring, strip, pad, pillow, sheet, tube, sphere, tablet or a bead covered by said foam.

Drug-containing foams can be utilized as stand-alone drug delivery platforms wherein the drug is incorporated into and is a part of the foam, or they can be used as one component of a more complex drug delivery system which may also comprise a suppository, tampon, or tampon-like device. The drug can be incorporated into the composition before foam formation of solid, semi-solid, or liquid structure, or it can be incorporated by partially or totally coating of the inner pores or a surface of a prefabricated polymeric foam scaffold.

A preferred route to deposit the drug would be to spray the foam with a concentrated drug solution, followed by drying of the solvent.

Drugs and other additives can be added to a lyophilized foam by spraying the foam with a dilute solution of the drug or additive in methylene chloride or ethanol. Preferably the quantity of solution, the temperature and the ambient air velocity are such that the solvent evaporates immediately after the solution is absorbed within the foam. This process is similar to that used when applying coatings to pills.

The volume of solution applied per gram of foam should be selected so that a substantial portion of the foam is coated. Having determined the appropriate solution volume, drug concentration is selected so that the desired drug dose per unit weight or per unit volume is obtained.

Alternatively, and less preferably, the drug solution can be metered by a nozzle onto the foam. This method may give less uniform coverage and slower solvent removal that the spraying method described above.

4. Release of the Drug From the Foam

In use, the preformed foam device is placed in a close contact with the epithelium in the nasal, oral, vaginal cavity or covering the labia and scrotum or the foam is formed in situ at the desired site of administration using a suitable composition that generates a porous foam structure immediately after administration, for example using sprayable or gellable compositions. The time of contact is determined by the desired therapeutic action of the drug and the release profile of the agents from the foam composition. Most preferred contact with the epithelium is at least two hours following in vivo placement. Optimal release of pharmacologically active agents can be attained up to 72 hours by the teachings of this invention. Longer drug release is possible by utilizing mixtures of polymers and/or additives permitting a long-term sustained extended drug release.

The release profiles are controlled by varying the composition of incorporated polymers and other additives, which affect porosity and density as well as by varying size of the device as will be apparent to those skilled in the art. Biodegradable foam systems begin to disintegrate into smaller units upon interaction with components at the site of administration. As the breakdown of the device occurs, drug is released from the foam following immediate, controlled or pulsed release kinetics.

Preferably, the active ingredient is continuously released for at least 8 hours after contact with the epithelium. Pulsed release can be desired for the first few hours, followed by a slower “maintenance” release rate up to 72 hours. Similar delivery profiles of drugs may be achieved using non-biodegradable foam systems whereas the rate of delivery of the pharmacologically active agent to or through the epithelial tissue is predominantly controlled by dissolution.

The device of the invention has good adhesive properties to maintain close contact to the epithelium at the site of administration. Adhesion may require interaction of polymeric compositions in this device with components at the site of administration such as water or ions.

Alternatively, foam compositions in the inventions may contain excipients that promote inherent adhesive properties of the device after administration. Adhesion of the device permits secure positioning of the device when worn and assures desired delivery of the active agent over the time frame beneficial to the therapy of the disease.

The active ingredient can primarily affect the surface of the epithelium where administered, which results in topical or local treatment of a disease or, alternatively, the primary effect occurs at a therapeutic target that is distinctly separated from the site of administration and, therefore, relies on systemic distribution of the active agent following transfer across the epithelial tissue into the systemic circulation. Upon contact with the mucus layer covering the vaginal epithelium, the lyophilizied foam first adsorbs fluid, which initiates the release of the active agent by dissolution and, simultaneously, supports the degradation process of the foam structure into a gel that possesses good structural integrity to deliver sumatriptan for a prolonged period prior to further dissolution into a liquid. This feature facilitates adhesion of the device and helps to control the rate of delivery of the active ingredient.

The time required for the devices of the invention to attain substantial dissolution to a liquid up to a point when the foam or film device structure is no longer evident is called the dissolution time and can be determined using in vitro dissolution techniques. At the time of complete dissolution, the biodegradable foam has completely dispersed as smaller polymer units within the nasal secretion, saliva or vaginal fluid. Therefore, there is no need to remove the device and normal excretion from the nasal, buccal or vaginal cavity will be completed by the continuous flow of physiological vaginal secretion.

A dissolution pattern and water uptake are seen in FIG. 3. Drug release from the foams or films of the inventions is controllable and may be changed by design. Specifically, certain polymers permit faster water uptake into the foam or gel resulting in faster release of the drug. Other polymers or mixtures, particularly those containing hydroxypropyl methylcellulose contribute to a slower water uptake and a decreased rate of the drug release. Water uptake rate is one indicator of the ability of a foam to release a drug. To determine the water uptake rate from foams, microcrystalline cellulose (Avicel) and HPMC, alone or in combination, were evaluated. Foams were prepared for this study according to Examples 5-7.

B. Film Compositions

In one embodiment, the invention concerns a polymer formulated into a film for topical or transepithelial vaginal, buccal, nasal, labial or scrotal delivery of therapeutic agents. The polymer films of the invention are high-surface-area sheets that are prepared from a variety of polymer solutions which are processed into a film.

Similarly to the foams, films of the invention are characterized by their controlled chemical and physical properties that can be tailored according to their intended application. Tuneable properties include hydrophilicity, rate of fluid absorption and degradation profile including a dissolution rate. The films of the invention thus release the active ingredient by dissolution or erosion or a combination of these mechanisms which may depend on interaction of the film composition with components at the site of administration, including but not limiting to fluid and ions. This will attain desired bioadhesive properties of the film and control the release rate of the agent as required by the therapeutic regimen for hours or days.

Typically, the film is prepared by dissolving an appropriate polymer, preferably a hydrophilic polymer, or a mixture thereof serving as a substrate material, as listed below, in an amount needed to prepare a solution of from about 1 to about 10% (w/w), in an aqueous or non-aqueous solvent, such as methanol, ethanol, glycerine, methylene, chloride, propylene glycol, propylene carbonate, glycofurol, cetyl alcohol, difluroethane and isopropyl alcohol, preferably purified water. A selected pharmaceutical agent or mixture of two or more such agents in an appropriate amount from about 0.01 to about 2000 mg and occasionally more, is then dissolved in an aqueous or non-aqueous solvent, preferably a purified water. Both solutions are mixed-together for from about 10 minutes to about several hours, preferably about 15-60 minutes, said mixture is spread over the flat surface or plate, such as a glass plate in a layer from 0.5 to about 2 mm, preferably about 1 mm, using, for example, a TLC coater and let dry at 25° C. for as long as it takes for the water to completely evaporate. The film layer typically dries in about 24 to about 148 hours, usually in about 70 hours. Alternatively, the film may be prepared by spraying said mixture and drying.

In alternative embodiments, polymeric solutions with the drug and additives may be prepared in acetic acid, cyclohexane, acetonitrile, tert-butanol, ethanol, and isopropanol or in mixtures of aqueous and non-aqueous solvents.

1. Single Layer Films and Multiple-Layer Films

Single-layer films containing drugs would be particularly useful applications where the film is in contact with tissue on both sides. Thus the drug would be able to diffuse out from both sides of the film.

Two-layer or more than two-layer films will be useful when a distinct function is required from the second layer. For example, for buccal applications, a drug-eluting layer is most desirable against the mucous membrane. On the opposite side, however, a second barrier film layer may be useful to prevent loss of the drug into the saliva and the digestive system. Useful barrier film polymers include polyethylene terephthalate, polyethylene, and nylon.

As a functional example of a multi-layer film, a multi-layer film would consist of a barrier film as described above, a middle layer which serves as the primary reservoir for the drug, and a third layer comprising mucoadhesives and/or release modifiers, which contacts the body and controls the adhesion of the film to the tissue and the rate at which the drug is released from the reservoir layer.

2. Film v. Foam Compositions

A polymer film is a uniform layer of material, usually less than 4 mm thickness, composed at least partly of a polymer which provides structural integrity. A film can optionally have a multilayer structure where each layer has a distinct composition. Normally the entrapped air in a film will be much less than 10% by volume. Thicker polymer layers up to 0.5 inches thick are usually referred to as sheets.

For the films of the current invention, the production method is to create a solution of at least one polymer. This solution can contain additional soluble and non-soluble polymers, drugs, transcutol, excipients, etc. The solution can be uniformly spread or sprayed over a flat surface (glass, paper, or another polymer sheet) and allowed to dry under ambient conditions or optionally with some heat. After the solvent evaporates, a film remains which can be peeled off. Films, due to their thinness, provide good patient comfort for nasal, buccal, vaginal, labial or scrotal applications.

In contrast, a polymeric foam may consist of a polymer composition, as described above, which contains at least 10%, and usually greater than 50%, void volume filled by air or another gas. For lyophilized foams, one starts with a solution of polymers and additives. Normally at least one polymer is water-soluble. After pouring the solution into molds of the desired shape, the solution is frozen solid. The frozen solutions, optionally after removal from the molds, are lyophilized at a low temperature, e.g. −40° C., and at low pressure until the water content has been reduced to a low level. After warming the samples under dry conditions, lyophilized foams in the shape of the mold are obtained. Foams are soft three-dimensional devices which can be particularly convenient for vaginal and labial treatments.

C. Substrate Materials for Producing Foam or Film Compositions

Substrate materials for preparation of foam or film compositions of the invention are polymers, hydrophilic or hydrophobic, preferably hydrophilic polymers. These polymers may be used singly or in combination with each other. They may be used in variable concentrations and ratio to each other when in admixture of two or several polymers.

Non-exclusive list of substrate polymers comprises cellulose and cellulose derivatives, microcrystalline cellulose, polyacrylic acid, polyethylene glycol, polypropylene glycol, divinyl glycol, polyethylene oxide, polypropylene oxide. Other possible polymers include the cellulose derivatives such as carboxymethyl cellulose, hydroxyethyl cellulose, polylactide, polyglycolide, polymethacrylic acid, poly-γ-benzyl-L-glutamate, polypropylene fumarate, poly-ε-caprolactone, poly-butylene terephthalate, polyvinyl alcohol, polyvinyl ether, poly-1-vinyl-2-pyrrolidinone, 2,5-dimethyl-1,5-hexadiene, divinyl benzene, polystyrene-divinyl benzene, polyanhydrides such as polybisp-carboxy-phenoxypropane-co-sebacic acid, polyhydroxyalkanoates such as poly-β-hydroxybutyrate or poly-β-butyrolactone, and alkyl-substituted silica gel such as tetraethylorthosilicate and dimethyldiethoxysilane.

1. Hydrophilic Polymers

Examples of hydrophilic polymers suitable for a foam or film manufacture include hydroxypropyl methylcellulose (HPMC), sodium carboxymethylcellulose, polyethylene glycol (PEG), alginic acid, alginic acid sodium salt, pectin, gelatin, collagen, polyvinyl pyrrolidone, poloxamer, acrylic-acid based polymers, such as carbopol, noveon, polyurethanes, polyvinyl alcohol, chitosan, hydroxypropyl cellulose, polyethylene oxide, fibronectin, hyaluronic acid, polysaccharide gums such as karaya gum, polyacrylamide, polycarbophil, dextran, xanthan gum, polyacrylamide, polyacrylamide, crosslinked polymethyl vinyl ether-co-maleic anhydride, commercially available as Gentrez™, gelatin, corn starch and mixtures thereof.

2. Hydrophobic Polymers

Examples of hydrophobic polymers suitable for formation of the foam and or film are, among others, polypropylene oxide,. polyamides, polystyrene, and polymethacrylic acid.

Examples of suitable and preferred substrate materials and mixtures thereof for preparation of foams and films are listed in Table 1.

TABLE 1

| | Composition | |
|---|---|---|
| Polymers | (% polymer) | Form |
| HPMC | 1.0 | Films |
| | 2.5 | Films |
| | 5.0 | Films |
| Gelatin | 1.0 | Films |
| | 2.5 | Films, Rods |
| | 5.0 | Films, Rods |
| | 10.0 | Films, Rods |
| Gelatin/HPMC | 1.0 | Films |
| (50/50) | 2.5 | Films |
| | 5.0 | Films |
| | 10.0 | Films |
| Alginic Acid | 1.0 | Films |
| | 2.5 | Films, Rods |
| | 5.0 | Films, Rods |
| | 10.0 | Films |
| Alginic Acid/HPMC | 1.0 | Films |
| (50/50) | 2.5 | Films |
| | 5.0 | Films |
| Alginic Acid/PEG 400 | 5.0 | Films, Rods |
| (25/75) | | |
| Alginic Acid/PEG 1400 | 5.0 | Films, Rods |
| (25/75) | | |
| Alginic Acid/PEG 4000 | 5.0 | Films, Rods |
| (25/75) | | |
| Alginic Acid/PEG 400 | 5.0 | Rods |
| w/Ketoconazole | | |
| (25/75) | | |
| Carbopol | 0.5 | Films |
| | 1.0 | Films |
| | 2.5 | Films |
| Noveon | 0.5 | Films |
| | 1.0 | Films |
| | 2.5 | Films |
| Pectin | 1.0 | Films |
| | 2.5 | Films, Rods |
| | 5.0 | Films, Rods |
| | 10.0 | Rods |
| Pectin/HPMC | 1.0 | Films |
| (50/50) | 2.5 | Films |
| | 5.0 | Films |
| Collagen | 0.5 | Films |
| | 1.0 | Films |
| | 2.5 | Films |

Alginic acid used is alginic acid sodium salt.

3. Additives

Foam and film formulations can comprise solely of two components, namely the polymer described above and the therapeutic agent described below in section D, or they can contain additional components including a variety of excipients and additives, such as release modifiers, mucoadhesive agents, and/or penetration enhancers/sorption promoters, fillers, dyes, etc., or other pharmaceutically acceptable excipients and additives.

a. Mucoadhesive Agents

As described above, the foam or film compositions of the invention contain a polymer, which may or may not have mucoadhesive properties. In many cases, the polymer, particularly a hydrophilic polymer, has a certain degree of mucoadhesive properties. Such properties advantageously support ability of the composition of the invention to adhere to the mucosal, labial or scrotal epithelium, however, it may or may not be sufficient to achieve the complete mucoadhesion for local adherence of the composition to the tissue or provide a sufficient support for a transepithelial, translabial or transscrotal delivery of the pharmaceutical agents. In such a case, the composition may conveniently contain still another mucoadhesive agent to achieve the prolonged and close contact with the tissue, adhesion of the composition to the tissue and interaction of the drug with the mucosal, labial or scrotal surface.

The mucoadhesive agent used to increase the adhesion of a film or foam device to a mucous membrane is preferably a polymer such as hydroxypropyl methylcellulose, carboxymethylcellulose, polylactide-co-glycolide, chitosan, chitosan ester or trimethylene chloride chitosan, sodium alginate, poloxamer, carbopol, pectin, or another cellulose derivative. Hydroxypropyl methylcellulose (HPMC) is particularly preferred for use in the present invention as it can be one of the substrates for preparation of the foam or film. Other examples of mucoadhesive agents include polyacrylic acid, hyaluronic acid, polyvinyl alcohol, polyvinyl pyrrolidone, polycarbophil and carbopol.

The mucoadhesive agent is typically present in from about 0.5 to about 10%.

b. Penetration Enhancers/Sorption Promoters

For delivery of drugs into the systemic circulation using transmucosal, translabial or transscrotal compositions, the composition additionally comprises a sorption promoter or penetration enhancer.

Sorption promoters or penetration enhancers are either ionizable or non-ionizable molecules that alter physical and/or biochemical barrier properties of the epithelia resulting in enhanced transfer of pharmacologically active agent to the systemic circulation.

Ionizable permeation enhancers include cationic, anionic, and zwitterionic excipients that are suitable to improve transfer of hydrophilic and lipophilic drug molecules across covering epithelia of the vaginal, nasal, oral cavity and labial or scrotal surfaces.

Preferred anionic permeation enhancers include derivatives of fatty acids, bile acids, phosphoric acid esters, carboxylates, and sulfates/sulfonates. For simplicity, sodium counterion is shown for anionic permeation enhancers, which is not limiting and includes any other biocompatible counterion that is currently known to the skilled in the art or will be discovered in the future.

Specifically, preferred anionic permeation enhancers include sodium caproate, sodium caprylate, sodium caprate, sodium laurate, sodium myristate, sodium palmitate, sodium palmitoleate, sodium oleate, sodium ricinoleate, sodium linoleate, sodium stearate, sodium lauryl sulfate, sodium tetradecyl sulfate, sodium laryl sarcosine, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodexoycholate, sodium glycol chenodesoxycholate, sodium cholylsarcosine, sodium N-methyl taurocholate, sodium tauro-24,25-dihydrofusidate, disodium polyoxyethylene-10 oleyl ether phosphate, esterification products of fatty alcohols or fatty alcohol ethoxylates with phosphoric acid or anhydride, ether carboxylates, succinylated monoglycerides, sodium stearyl fumarate, steaoryl propylene glycol hydrogen succinate, mono/diacetylated tartaric acid esters of mono- and diglycerides, citric acid esters of mono- and diglycerides, glyceryl-lacto esters of fatty acids, lactylic esters of fatty acids, alginate salts, ethoxylated alkyl sulfates, alkyl benzene sulfones, a-olefin sulfonates, acyl isethionates, acyl taurates, alkyl glyceryl ether sulfonates, octyl sulfosuccinates disodium, disodium undecylenamideo-MEA-sulfosuccinate, phosphatidic acid, phosphatidyl glycerol, polyacrylic acid, hyaluronate sodium, glycyrrhetinic acid, ethylene diamine tetraacetate and sodium citrate.

Cationic permeation enhancers include ammonium and pyridinium salts. For simplicity, chloride conterion is shown for cationic permeation enhancers, which is not limiting and includes any other biocompatible counterion that is currently known to the skilled in the art or will be discovered in the future. Specifically, preferred cationic permeation enhancers include chitosan, trimethyl chitosan, poly-L-arginine chitosan, poly-L-lysine chitosan, aminated gelatin, hexadecyl triammonium chloride, decyl trimethylammonium chloride, cetyl trimethylammonium chloride, alkyl benzyldimethylammonium chloride, diisobutyl phenoxyethoxydimethyl benzylammonium chloride, ethyl pyridinium chloride, isopropyl pyridinium chloride, N-lauryl,N,N-dimethylglycine, N-capryl,N,N-diethylglycine, polyoxyethylene-15 coconut amine, poly-L-lysine, poly-L-arginine.

Zwitterionic permeation enhancers include naturally occurring and synthetic compounds that exhibit simultaneous positive and negative charges at the site of administration. Specifically, preferred zwitterionic permeation enhancers include lecithin, lysolecithin, hydroxylated lecithin, lysophosphatidylcholine, phosphatidylcholine, phosphatidylethanolamine, phosphatidylserine, didecanoyl-L-α-phosphatidylcholine, laurolylcarnitine, acylcarnitine, palmitoyl-D,L-carnitine.

Concentration of these enhancers varies significantly from compound to compound, however, they are preferably used in concentration from about 0.01 to about 60%, and more preferably from about 10 to about 15%.

Non-ionizable glycol ether derivative is a polyoxyethylene alkyl ether, ester or a glycol derivative with glycerol ester represented by a compound selected from the group consisting of polyoxyethylene alkyl ether such as, for example, polyoxyethylene lauryl ether, polyoxyethylene monooleyl ether and ethoxydiglycol, polyoxyethylene alkyl phenol, such as, for example polyoxyethylene nonylphenol and polyoxyethylene octylphenol ether, polyoxyethylene sterol, such as, for example polyoxyethylene cholesterol ether and polyoxyethylene soya sterol ether and cyclodextrins, such as, for example, α-cyclodextrin, β-cyclodextrin, γ-cyclodextrin, dimethyl-β-cyclodextrin, methylated-β-cyclodextrin, 2-hydroxypropyl-β-cyclodextrin and sorbitol.

Non-ionizable glycol ester derivative is a polyoxyethylene glycol ester, polyoxyethylene glycerol fatty acid ester, polyoxyethylene glycerol fatty acid ester, polyoxyethylene glyceride or polyoxyethylene vegetable or hydrogenated oil, said derivative represented by a compound selected from the group consisting of polyoxyethylene glycol ester, such as, for example, polyoxyethylene monooleate, polyoxyethylene dilaurate, polyoxyethylene mono and dioleate, polyoxyethylene glycerol fatty acid ester, such as, for example, polyoxyethylene glyceryl laurate and polyoxyethylene glyceryl oleate, polypropylene glycol fatty acid ester, such as, for example, propylene glycol oleate and propylene glycol stearate, polyoxyethylene glyceride, such as, for example, polyoxyethylene sorbitan monooleate and polyoxyethylene tristearate, polyoxyethylene vegetable or hydrogenated oil, such as, for example, polyoxyethylene hydrogenated castor oil, polyoxyethylene almond oil, polyoxyethylene apricot kernel oil, polyoxyethylene caprylic or capric glyceride and lauroyl macrogol glyceride.

Non-ionizable glycol derivative with glycerol ester is represented by glycol derivative with glycerol ester, such as, for example, polyoxyethylene oleate and polyoxyethylene glyceryl stearate.

In polymer compositions used for formation of foam or films according to the invention, the variable or non-ionizable enhancers are present in an amount from about 0.01 to about 60%, preferably from about 5 to about 25%, most preferably from about 10 to about 15%, by weight.

The most preferred non-ionizable glycol derivative is ethoxydiglycol, also known as TRANSCUTOL®, commercially available from Gattefosse, Westwood, N.J.

c. Release Modifiers

In order to achieve desirable drug release from the mucosal, transmucosal, labial, translabial, scrotal, or transscrotal foam or film compositions, the pharmaceutical agent is optionally incorporated into a vehicle or carrier for which the drug has low affinity and which promote a drug release from the foam or film or which can modify a rate of such release. Hence, lipophilic drugs are incorporated into hydrophilic modifiers and lipophilic drugs are incorporated into hydrophilic carriers.

Hydrophilic modifiers include polyethylene glycol 200, polyethylene glycol 8000, poloxamer, polyoxyethylene glycerylcocoate and carbopol.

Hydrophilic modifiers include polyethylene glycol 200, polyethylene glycol 8000, poloxamer, polyoxyethylene glycerylcocoate and carbopol.

Hydrophobic modifiers include Suppocire AS2, Suppocire AS2X, suppocire CM, Witepsol H15, Witepsol W25, mineral oil, corn oil, paraffin oil, canola oil, castor oil, cottonseed oil, lecithin, peanut oil, sesame oil, soybean oil and hydrogenated vegetable oil.

Release modifiers may be present in the composition in the amounts from about 5% to about 70% by weight.

d. Additional Excipients and Additives

1. Solubilizing Agents

Solubilizing agents are used to increase the solubility of an agent in a formulation during the production of a device or, alternatively, to increase the solubility of an agent in fluids of tissue during the use of a device.

Any pharmaceutically acceptable solubilizing agent may be used. Preferred solubilizing agents are polyethylene glycol (PEG), cyclodextran, glycofurol, propylene glycol, propylene carbonate and surfactants.

Solubilizing agents are typically added in amount from about 5% to about 30%.

2. Buffering Agents

Buffering agents are used for control of the pH of the immediate environment of the device in order to control or enhance the release of an agent. Any pharmaceutically acceptable buffering agent or a mixture thereof may be used for the purposes of this invention. Exemplary buffering agents are potassium metaphosphate, potassium phosphate, monobasic sodium acetate, sodium carbonate, sodium bicarbonate, boric acid, tartaric acid, tris citrate and triethanolamine.

Buffering agents are typically added in amount from about 1% to about 10%.

3. Fillers

Fillers are inert ingredients used to increase the size or improve the usability of a device. Any pharmaceutically acceptable filler may be conveniently used for the purposes of this invention. Exemplary fillers are calcium carbonate, silicon dioxide, titanium dioxide, paraffin, stearic acid, talc, wax and zinc stearate.

Fillers are typically added in amount from about 5% to about 15%.

4. Preservatives

Preservatives are used to prevent the growth of microorganisms during storage. All pharmaceutically suitable preservatives may be used. The preferred preservatives are benzalkonium chloride, propyl paraben, benzyl alcohol, sorbic acid, phenol, phenylethyl alcohol, BHA and BHT.

Preservatives are typically added in amounts from about 0.01% to about 5%.

5. Plasticizers

Plasticizers are compounds used to soften the film or foam. Exemplary plasticizers are glycerin, water, polyethylene glycol, propylene glycol, sorbitol and triacetin, to name a few.

Plasticizers are typically added in amount from about 5% to about 25%.

6. Surfactants

Surfactants, such as Tween 80, sodium lauryl-sulfate and Brij, may be advantageously added as needed in amount from 0.01% to about 5%.

7. Antioxidants

Antioxidants suitable to be used for foams and films are selected from ascorbic acid, BHA, BHT, sodium bisulfite, vitamin E, sodium metabisulphite and propyl gallate and may be added in amounts from 0.1% to about 3%.

D. Pharmacological Agents

Foam or film compositions of the invention are suitable for topical or transepithelial delivery of any pharmacological agent or a mixture of two or more agents which asserts a therapeutic effect when delivered locally to vaginal, nasal, buccal, labial or scrotal epithelium or can be delivered to the systemic circulation through the vaginal, nasal, buccal, labial or scrotal epithelium.

a. Representative Pharmacological Agents

Representative pharmacological agents which may be conveniently delivered using foams or films of this invention are groups of anti-inflammatory agents, calcium or potassium channel antagonists, β-adrenergic agonists, vasodilators, topical anesthetics, cyclooxygenase inhibitors, antimicrobial, antiviral, antipsychotic, anti-epileptic, antifungal, anti-osteoporotic, anti-migraine, anti-HIV, anti-neurodegenerative, anti-cancer agents, opioid analgesics, and biotechnology-derived pharmacological agents, such as proteins and peptides.

Non-limiting representative examples of these drugs are nonsteroidal anti-inflammatory drugs which include aspirin, ibuprofen, indomethacin, diclofenac, phenylbutazone, bromfenac, fenamate, sulindac, nabumetone, ketorolac, and naproxen.

Examples of calcium channel antagonists include diltiazem, israpidine, nimodipine, felodipine, verapamil, nifedipine, nicardipine, and bepridil.

Examples of potassium channel blockers include dofetilide, almokalant, sematilide, ambasilide, azimilide, tedisamil, sotalol, piroxicam, and ibutilide.

Examples of β-adrenergic agonists include terbutaline, salbutamol, metaproterenol, and ritodrine.

Vasodilators include nitroglycerin, isosorbide dinitrate and isosorbide mononitrate.

Examples of cyclooxygenase (COX) inhibitors are acetylsalicylic acid, naproxen, ketoprofen, ketorolac, indomethacin, fenamate, ibuprofen, diclofenac, tenoxicam, bromfenal, celecoxib, nabumetone, phenylbutazone, rofecoxis, sulindac, meloxicam and flosulide.

Examples of local anesthetics include lidocaine, mepivacaine, etidocaine, bupivacaine, 2-chloroprocaine hydrochloride, procaine, and tetracaine hydrochloride.

Examples of anti-osteoporotic drugs are bisphosphonates selected from the group consisting of alendronate, clodronate, etidronate, pamidronate, tiludronate, ibandronate, alpadronate, residronate, neridronate and zoledronic acid.

Examples of antifungal, antimicrobial drugs are miconazole, terconazole, isoconazole, fenticonazole, fluconazole, nystatin, ketoconazole, clotrimazole, butoconazole, econazole, metronidazole, clindamycin, 5-fluoracil, acyclovir, AZT, famovir, penicillin, tetracycline, erythromycin, amprenavir, amividine, ganciclovir, indivaris, lapinavis, nelfinavir, rifonavir and saguinar.

Examples of anti-migraine drugs are almotriptan, eletriptan, flavotriptan, naratriptan, rizatriptan, sumatriptan, zolmitriptan, ergotamine, dihydroergotamine, bosentan and lanepitant.

Examples of anti-neoplastin or chemotherapeutic drugs are vincristine, cisplastin, doxorubicin, daunorubicin, actinomycin D, colchicin, digoxin, etoposide, topotecan, irinotecan, paclitaxel, docetaxel, cyclophosphamide, methotrexate, gemcitabine, mitoxantrone, topotecan, teniposide, vinblastine and mytomycin C.

Examples of anti-HIV drugs are saquinavir, ritonavir, indinavir, amprenavir, nelfinavir, lopinavir and ganciclovir.

Examples of antinausea drugs are aprepitant, cyclizine, dolasetron, domperidone, dronabinol, levonantradol, metoclopramide, nabilone, ondansetron, prochlorperazine, promethazine and tropisetron.

Examples of opioid analgesics are buprenorphine, dynorphin A, fentanyl, Met-enkaphalin, morphine, naloxone, pentazosine and spiradoline.

Examples of antiepileptic drugs are carbamazepine, clonazepam, phenobarbital, phenytoin, primidone, andvalproate.

Examples of anti-psychotic drugs for treatment of neurogenerative diseases are bromocriptine, carbidopa, galantamine, memantine, pergolide, selegiline, tacrine and trihexyphenidyl.

Examples of drugs for treatment of psychiatric disorders are alprazolam, amitriptyline, amoxapine, bupropion, buspirone, chlordiazepoxide, chlorpromazine, clozapine, diazepam, fluoxetine, fluphenazine, haloperidol, imipramine, loxapine, metrotiline, oxazepam, paroxetine, perephenazine, phenelzine, pimozide, prazepam, protriptyline, risperidone, selegiline, sertraline, thoridazine and trazodone.

Examples of antinausea drugs are aprepitant, cyclizine, dolasetron, domperidone, dronabinol, levonantradol, metoclopramide, nabilone, ondansetron, prochlorperazine, promethazine and tropisetron.

Examples of biotechnology-derived drugs are insulin, calcitonin, somatostatin, vasopressin, luprolide, oxytocin, bivalirudin, integrilin, natrecor, abarelix, gastrine G17 peptide, ziconotide, cereport, interleukins, humanized antibodies and growth hormone.

b. Doses of Pharmacological Agents

Pharmacological agents are added in amount which is therapeutically effective locally or systemically. Typically, the drug will be added in amount from about 0.01 to about 2000 mg as shown below. Occasionally, the dose may exceed 2000 mg range up to 20,000 mg, particularly when there is a repeated administration.

Calcium channel antagonists: bepridil (50-1600 mg), diltiazem (30-1500 mg), felodipine (1-50 mg), israpidine (1-20 mg), nicardipine (30-600 mg), nifedipine (15-650 mg), nimodipine (100-1400 mg), verapamil (100-1500 mg).

Potassium channel blockers: almokalant, ambasilide, azimilide, dofetilide (0.2-5 mg), ibutilide (0.3-5 mg), sematilide, sotalol, (80-1300 mg), tedisamil.

β-Adrenergic agonists: metaproterenol (20-240 mg), ritodrine (100-2000 mg), salbutamol (0.1-5 mg), terbutaline (1-60 mg).

Vasodilators: isosorbide dinitrate (10-500 mg), isosorbide mononitrate (10-250 mg), nitroglycerin (2-150 mg).

Cyclooxygenase inhibitors: acetylsalicylic acid (5-8000 mg), bromfenac, celecoxib (100-2400 mg), diclofenac (50-800 mg), fenamate, flosulide, ibuprofen (600-6,000 mg), indomethacin (30-600 mg), ketoprofen (50-1200 mg), ketorolac (5-200 mg), meloxicam (2-60 mg), nabumetone (500-4,000 mg), naproxen (100-3000 mg), phylbutazone, rofecoxib (5-200 mg), sulindac, tenoxicam.

Local anesthetics: 2-chloroprocaine (50-2400 mg), bupivacaine (50-1600 mg), etidocaine, lidocaine (10-150 mg), mepivacaine (25-1600 mg), procaine (150-3,000 mg), tetracaine.

Anti-osteoporotic drugs: alendronate (2-160 mg), alpadronate, clodronate (1-3200 mg), etidronate (2-1400 mg), ibandronate (0.01-100 mg), neridronate (0.1-200 mg), pamidronate (1-3,000 mg), residronate (0.05-50 mg), tiludronate (0.02-400 mg), zoledronic acid (0.05-150 mg).

Antimicrobial drugs: acyclovir (100-4,000 mg), amprenavir (150-7,200 mg), amivudine (10-1200 mg), butoconazole, clindamycin (75-20,000 mg), clotrimazole (5-200 mg), econazole (2-100 mg), erythromycin (100-16,000 mg), famovir, fenticonazole, fluconazole (50-1600 mg), ganciclovir (250-12,000 mg), indinavir (400-9,600 mg), isoconazole, ketoconazole (1-6400 mg), lopinavir (50-2000 mg), metronidazole (100-10,000 mg), miconazole (600-15,000 mg), nelfinavir (300-10,000 mg), nystatin (0.5-12 Mio U), penicillin VK (100-8000 mg), ritonavir (150-4800 mg), saquinavir (300-15,000 mg), terconazole (2-400 mg), tetracycline (300-16,000 mg).

Antimigraine drugs: almotriptan (2-100 mg), bosentan (50-1000 mg), dihydroergotamine (1-20 mg), eletriptan (1-400 mg), ergotamine, flavotriptan, lanepitant, naratriptan (0.5-20 mg), rizatriptan (2-120 mg), sumatriptan (10-800 mg), zolmitriptan (0.5-40 mg).

Antineoplastic/Chemotherapeutic drugs: actinomycin D, cisplatin (5-400 mg/m²), colchicin (0.1-50 mg), cyclophosphamide (50-800 mg), daunorubicin, docetaxel, doxorubicin (50-2,500 mg/m²), etoposide, gemcitabine (70-4,000 mg/ml²), irinotecan, methotrexate (0.2-40 mg), mitoxantrone (0.05-2 mg/m²), mytomycin C, paclitaxel, teniposide, topotecan, vinblastine, vincristine (1-200 mg).

Biotechnology-derived drugs: abarelix, bivalirudin (0.5-1000 mg), calcitonin (100-20,000 IU), cereport, gastrine G17 peptide, growth hormones, humanized antibodies, insulin, integrilin (0.1-1400 mg), interleukins, luprolide, natrecor (0.001-2 mg), oxytocin (0.01-10,000U), somatostatin, vasopressin (0.1-40,000U), ziconotide.

Antinausea drugs: aprepitant (40-600 mg), cyclizine, dolasetron (25-400 mg), domperidone, dronabinol (1-60 mg/m²), levonantradol, metoclopramide (10-200 mg), nabilone, ondansetron (4-75 mg), prochlorperazine (5-600 mg), promethazine (5-200 mg), tropisetron.

Opioid analgesics: buprenorphine (0.5-2000 mg), dynorphin A, fentanyl (0.1-10 mg), met-enkephalin, morphine (30-1000 mg), naloxone (0.1-3000 mg), pentazocine (50-1500 mg), spiradoline.

Antiepileptic drugs: carbamazepine (100-9,600 mg), clonazepam (3-60 mg), phenobarbital (15-800 mg), phenytoin (150-1200 mg), primidone (5-3000 mg), valproate (350-12,000 mg)

Drugs in neurodegenerative diseases: bromocriptine (0.5-400 mg), carbidopa (5-400 mg), galantamine (4-100 mg), memantine, pergolide (0.02-20 mg), selegiline (2-40 mg), tacrine (20-650 mg), trihexypehenidyl (0.5-40 mg)

Drugs in psychiatric disorders: alprazolam (0.2-40 mg), amitriptyline (5-400 mg), amoxapine (25-1200 mg), bupropion (25-1800 mg), buspirone (5-250 mg), chlordiazepoxide (5-1200 mg), chlorpromazine (10-3200 mg), clozapine (5-1200 mg), diazepam (1-200 mg), fluoxetine (5-350 mg), fluphenazine (0.2-40 mg), haloperidol (0.5-400 mg), imipramine (10-1200 mg), loxapine (10-1000 mg), maprotiline (10-1000 mg), oxazepam (20-600 mg), paroxetine (5-250 mg), perphenazine (10-300 mg), phenelzine (20-400 mg), pimozide (0.5-40 mg), prazepam, protriptyline (10-300 mg), risperidone (0.1-20 mg), selegiline (2-40 mg), sertraline (10-800 mg), thoridazine, trazodone (50-1200 mg.

c. Uniformity and Release of Pharmacological Agents from the Foam or Film Composition

In order to determine whether the foam or film of the invention is efficacious for the drug delivery and thus suitable for therapeutic purposes, release of the drug from the foam or film and its uniformity was determined.

Uniformity, expressed as % of recovery and release of pharmacological agents from the foam was determined using lyophilized foam rods comprising ketorolac tromethamine in alginic acid sodium salt.

The uniformity of the distribution of the ketorolac in the foams prepared according to Example 5 was measured by a UV absorbance method. A standard curve for ketorolac in deionized water was developed by measuring the UV absorbance at 322.5 nm (path length 12.31 mm) for alginic acid alone, for ketorolac solutions comprising ketorolac (7.4%) and alginic acid, sodium salt (92.6%), and ketorolac (3.8%), alginic acid (48.1%) and hydroxypropyl methylcellulose (48.1%) mixture. Alginic acid solution alone without the drug serving as a control had a negligible absorbance.

For this study, three foam rods A, B and C prepared from the mixture containing 7.4% ketorolac and 92.6% alginic acid, were selected for analysis. About 2 mm of irregular material was trimmed from both ends of the foam rods. Using a razor blade, each foam rod was divided into 5 shorter cylindrical sections of length 9 mm. The weight of each section was recorded. Each section was dispersed into 200 ml deionized water using a high intensity mixer. The UV absorbance at 322.5 nm was recorded for each solution.

From.the standard curve, the ketorolac concentration in ug/ml of solution was calculated from the following relationship: absorbance=0.051× Concentration+0.0001.

For each foam section, the concentration multiplied by 200 ml gives the weight (μg) of ketorolac in that section. For each section, the ketorolac weight is divided by the weight of the foam section to yield the ketorolac weight per section in pg ketorolac per mg of foam. Finally, the obtained result is divided by the ideal value from the formulation (73.4 ug/mg of foam) to give the % ketorolac recovered for each foam section. Results are seen in Table 2.

TABLE 2
Ketorolac Recovery (%)

| | Foam | Foam | Foam |
|---|---|---|---|
| Foam Section # | Rod A | Rod B | Rod C |
| 1 | 99.7 | 98.6 | 96 |
| 2 | 100 | 97.3 | 97.3 |
| 3 | 92.1 | 96.7 | 95.8 |
| 4 | 91.8 | 99.5 | 99 |
| 5 | 96 | 94.7 | 97.7 |
| Mean | 95.9 | 97.4 | 97.2 |
| Standard Deviation | 3.95 | 1.85 | 1.31 |
| High/Low Ratio | 1.09 | 1.04 | 1.03 |
| High/Low Ratio, | 1.09 | | |
| All Data | | | |

Ideal, 100%, recovery of ketorolac is 73.4 ug of ketorolac per 1 mg of foam.

Alginic acid sodium salt (AA) solution concentration contained 2.5 g of alginic acid per 100 g water.

Concentration of ketorolac tromethamine represented 7.43% of foam weight. Ratio of ketorolac:AA was 2:25.

As seen in Table 2, mean recovery for all three rods were very close to 100%, namely 95.7, 97.4 and 97.7%, respectively. Results show that almost 100% release of ketoroloc can be achieved from the foam prepared from alginic acid sodium salt when the drug is present in about 2:25 ratio of the drug to the polymer.

The above study was further expanded for release of ketorolac tromethamine from alginic acid sodium salt/HPMC foams in pH 4.22 phosphate buffer. For that study, ketorolac concentration was 7.4%, normalized to 120 mg foam. The foam was prepared from alginic acid sodium salt/HPMC mixture.

Results are seen in FIG. 1 which shows that the foam prepared from a mixture of ketorolac, alginic acid and HPMC has slower more controlled release of ketorolac than the one prepared from ketorolac and alginic acid only.

Results seen in FIG. 1 show that the foams prepared from mixtures of ketorolac, alginic acid sodium salt, and HPMC have slower more controlled release than the one prepared from ketorolac and alginic acid sodium salt only.

As seen in FIG. 1, approximately 93% of ketorolac was released from the alginic acid foam at 2 hours, while approximately 54% of the drug was released at the same time from the 50:50 AA:HPMC foam.

These results illustrate the point of a slow versus fast release of the drug from the foam. The speed of the release may be conveniently controlled and regulated by changing the substrate or by combining the substrate materials and varying their proportions relative to each other or relative to the drug.

The data further show that the distribution of ketorolac in the lyophilized alginic acid or alginic acid/HPMC mixture is extremely uniform.

As seen in FIG. 1, approximately 93% of ketorolac was released from the alginic acid foam at 2 hours, while approximately 54% of the drug was released at the same time from the alginic acid/HPMC foam (50:50).

These results illustrate the point of a slow versus fast release of the drug from the foam. The speed of the release may be conveniently controlled and regulated by changing the substrate or by or combining the substrate materials and varying their individual proportions relative to each other or relative to the drug.

The data further show that the distribution of ketorolac in the lyophilized alginic acid or alginic acid/HPMC mixture is extremely uniform.

The same type of experiment was performed for a film composition where the ketorolac release from the alginic acid film into a synthetic vaginal fluid at pH 4.2 was determined.

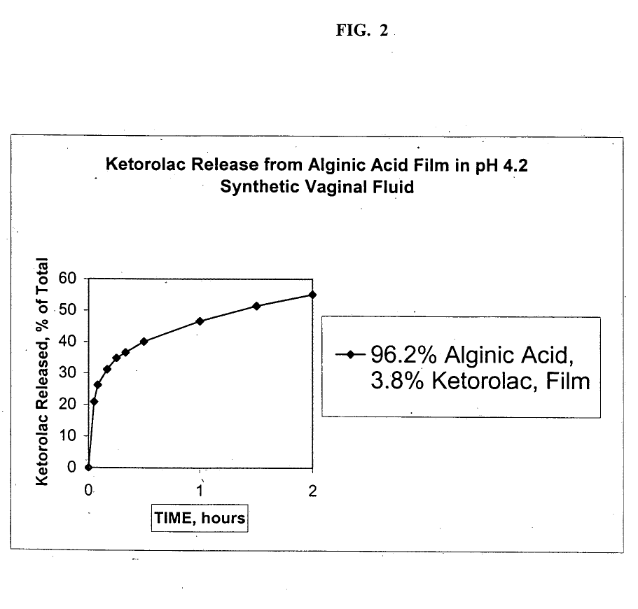

As seen in FIG. 2, at two hours interval, approximately 55% of ketorolac was released from the film prepared from a film prepared from a solution consisting of 96.2% alginic acid (sodium salt) and 3.8% of ketorolac. The film was prepared according to Example 7.

The same type of experiment was performed for a film composition where the ketorolac release from the alginic acid film into a synthetic vaginal fluid at pH 4.2 was measured. As seen in FIG. 2, after 2 hours approximately 55% of the ketorolac was released from a film prepared from a solution consisting of 96.2% alginic acid sodium salt and 3.8% of ketorolac. The film was prepared according to Example 7.

d. Drug Release from the Foam

Drug release from the foams or films of the inventions is controllable and may be changed by design. Specifically, certain polymers permit a fast water uptake into the foam or gel resulting in faster release of the drug, other polymers or mixtures, particularly those containing hydroxypropyl methyl cellulose contribute to a decreased rate of the drug release. To determine a water uptake and drug release from the foam, microcrystalline cellulose (AVICEL), HPMC, alone or in combination in various concentrations was tested. Foam prepared for this study were according to Examples 4-6.

Results of this study are shown in FIG. 3. FIG. 3 clearly shows that the foam prepared from the AVICEL/HPMC mixture (95.2%/4.8%) takes up water much faster and in larger amounts than the foam prepared from AVICEL/HPMC mixture containing the same amount of-each (50%/%50%) or foam prepared solely from HPMC.

FIG. 3 demonstrates that for foam prepared from AVICEL/HPMC mixtures, the water uptake depends on a proportion of microcrystalline cellulose (AVICEL). Faster water uptake is observed when the proportion relative to HPMC is higher. HPMC slows down the water uptake.

e. Modifying Drug Release

To fabricate foam or film layers with rapid release properties of the pharmacologically active agent, the polymer or mixture of polymers is selected to enhance solubility of the drug in the hydrated polymer layer. For high-solubility drugs, hygroscopic polymers such as cellulose derivatives are used alone or in combination with excipients that decrease viscosity, such as, for example, surfactants. Alternatively, dissolution of low-solubility drugs can be accelerated by incorporation of small fractions of hydrophobic polymers such as polyethylene or polypropylene and the use of solubility enhancers and/or surfactants.

Controlled or sustained release is achieved by incorporating polymers that increase viscosity upon hydration or polymers that decrease solubility of the drug. Incorporation of drug particles of different physical forms such as amorphous vs. crystalline can also delay the release of the drug from the foam or film device. Balanced approaches that include a combination of rapid with sustained release layers will achieve pulsed release that may be beneficial for the therapy of the disease.

The topical foams, films, and sprays typically contain a mucoadhesive agent in the amount of about 0.5% to about 10% concentration by weight, about 1% to about 10% penetration enhancer, and about 1% to about 10% buffering agent, wherein the drug to polymer ratio is from about 1-15 to about 85-99.

The transmucosal, translabial or transscrotal foams and films typically contain a mucoadhesive agent in the amount of about 0.5% to about 25% concentration by weight, about 5% to about 25% penetration enhancer and about 1% to about 10% buffering agent, wherein the drug to polymer ratio is about 1-15 to about 85-99.

Topical foams or films of the invention comprise at least of a hydrophilic or hydrophobic polymer, preferably a polymer which has a mucoadhesive properties and a pharmacological agent. If the mucoadhesive properties of the polymer are slight or if the polymer has no mucoadhesive properties, then the mucoadhesive agent is added.

Transmucosal drug delivery permits transport of the drug into the systemic circulation directly through the nasal, buccal, vaginal, labial or scrotal epithelium, thereby avoiding invasive intravenous or less effective oral administration.

II. Therapeutic Compositions

Therapeutical compositions of the invention are either topical nasal, buccal, vaginal, labial or scrotal compositions or transepithelial compositions delivering the drug to the systemic circulation through the nasal, buccal or vaginal mucosa or through the labial or scrotal epithelium.

d. Topical Nasal, Buccal, Vaginal, Labial or Scrotal Foams or Films

Topical foams or films of the invention comprise at least of a hydrophilic or hydrophobic polymer, preferably a polymer which has a mucoadhesive properties and a pharmacological agent. If the mucoadhesive properties of the polymer are slight or if the polymer has no mucoadhesive properties, then the mucoadhesive agent is added.

B. Transepithelial Compositions

Transepithelial drug delivery permits transport of the drug into the systemic circulation directly through the nasal, buccal and vaginal mucosa or through labial or scrotal epithelium, thereby avoiding invasive intravenous or less effective oral administration.

Transmucosal or trans-epithelial foams or films of the invention typically comprise at least of a hydrophilic or hydrophobic polymer substrate, preferably a polymer which has a mucoadhesive properties, penetration enhancer or sorption promoter and a pharmacological agent. If the mucoadhesive properties of the polymer are slight or if the polymer substrate has no mucoadhesive properties, then the additional mucoadhesive agent is added.

C. Specific Exemplarv Foam or Film Compositions

Specific and preferred topical, and transepithelial foam or film compositions are those comprising a polymer, preferably mucoadhesive polymer or a mixture of polymers formulated for rapid or slow drug delivery. These compositions and also include empty foams or films which can be conveniently incorporated with a drug solution or powder. Also included are compositions wherein the foam or film is used.for coating of conventional devices, such as tampons and, depending on the polymer(s) used for regulation of drug release form such devices, depending on their use.

Thus, for rapid drug release for topical use the composition contains mostly AVICEL-like polymers in combination with an appropriate mucoadhesive agent while for a slow release the composition will primarily contain HPMC-like polymers which may have mucoadhesive properties but primarily regulate the release of the drug.

Foam or film compositions of the invention consist essentially of a combination of an effective amount of a pharmacological agent from about 0.01 mg to about 2000 mg and occasionally higher, said agent selected from the group of agents exemplarily listed above in section D or any other drug suitable for transmucosal delivery, incorporated into a foam or film prepared from a polymer or mixture thereof and preferably containing at least one or several penetration enhancers and/or a release modifier and/or additional mucoadhesive agent and/or additional nontoxic pharmacologically acceptable biocompatible excipient.

Said composition is typically formulated as a foam or film suitable for insertion into a nasal, buccal or vaginal cavity or in a shape suitable for placement on the labia or scrotum, said composition further optionally incorporated into a nasal, buccal, vaginal, labial or scrotal device or covering such device.

Specific representative compositions are listed in Table 3.

TABLE 3
FOAM AND FILM FORMULATIONS

| | Ex. A | | Ex. B | | Ex. C | | Ex. D | | Ex. E | | Ex. F | | Ex. F-1 | |
|---|---|---|---|---|---|---|---|---|---|---|---|---|---|---|
| Material | Wt/g | Wt % | Wt/g | Wt % | Wt/g | Wt % | Wt/g | Wt % | Wt/g | Wt % | Wt/g | Wt % | Wt/g | Wt % |
| AA | 1.2503 | 46.3 | 2.5023 | 92.6 | 2.5 | 96.2 | | | | | | | | |
| HPMC | 1.2507 | 46.3 | | | | | 1 | 4.8 | 5.0014 | 50 | 5.0002 | 100 | 5.0044 | 20 |
| Ktr | 0.2015 | 7.46 | 0.2002 | 7.41 | 0.1 | 3.8 | | | | | | | | |
| Avicel | | | | | | | 20.192 | 95.2 | 5.005 | 50 | | | 20.0017 | 80 |
| Water | 100 | | 100 | | 50 | | 79 | | 90 | | 95 | | 75 | |
| Form | Foam | | Foam | | Film | | Foam | | Foam | | Foam | | | |
| | Ex. 5 | | Ex. 6 | | Ex. 7 | | Ex. 8 | | Ex. 9 | | | | Ex. 10 | |

AA = Alginic Acid, Sodium Salt (Sigma)
HPMC = Hydroxypropylmethyl Cellulose USP (Dow Chemical)
Ktr = Ketorolac Tromethamine USP (Quimica Sintetica)
Avicel = Avicel NF, Ph-101 (FMC Biopolymer), nominal particle size 50 microns
Wt % = Weight % of dry components in the foam

In a general method for preparing the transmucosal or trans-epithelial compositions of the invention, 0.01 to 2000 mg of the drug is dissolved in a solvent, aqueous or non-aqueous, depending on the nature of the drug and combined with a polymer or polymer mixture used for foam or film preparation and subjected to appropriate process to fabricate foams and films as described above, preferably lyophilization, aeration, spray-drying or drying as described above. Other additives, as described, may or may not be added. Resulting foam or film may be formed as a stand alone device or incorporated into a device, such as an intravaginal tampon, foam suppository, foam tablet, foam pessary, etc., or molded into a buccal dissolvable tablet, strip or patch or incorporated into a foam capsule, gel capsule or another form suitable for buccal, nasal insertion and suitable for these applications, or as described above, may be incorporated into or used for coating of an independent non-foam, non-film device.

Typically, for transepithial vaginal, labial and scrotal delivery, the composition will contain higher percentage of the mucoadhesive agent and penetration enhancer than for nasal or buccal transmucosal delivery as the barrier properties of the nasal and buccal mucosa are less restrictive and blood supply is closer to the mucosal surface than in the vaginal mucosa. For labial or scrotal use, the foam or film will contain the higher amount of the mucoadhesive agent and amount of penetration enhancer will also be generally higher as these compositions have to cross non-cornified or cornified non-mucosal epithelium.

The foam or film according to the invention compositions are useful for delivery of drugs by permeation through the vaginal, nasal, buccal, labial or scrotal epithelium directly to the systemic circulation. The mucoadhesive polymer enhances adhesion of the foam or film to the covering epithelia and the glycol derivative optimally present in these compositions enhances permeation through the mucosa, particularly of the drugs which would otherwise not be able to cross the nasal, oral, vaginal, labial or scrotal epithelial barrier.

Moreover, the drug compounds solubilized with a glycol derivative in combination with an appropriate mucoadhesive agent allow a prolonged contact of the drug with the mucosal surface, thereby further enhancing the efficiency of delivery of the compound.

III. Formulations and Devices

Each foam or film composition of the invention is formulated for its specific use, namely for the use as topical or transepithial vaginal, nasal, buccal or labial, translabial, scrotal or transscrotal foam or film.

A. Formulations

Formulations are prepared specifically for the intended use of delivery route.

Thus, for nasal transepithelial administration, the composition is formulated as a foam or film, preferably sprayable foam or gellable film.

For buccal transepithelial delivery, the composition is formulated as a foam tablet or capsule or gel foam or spray or is microincorporated into a device insertable into the buccal space, such as a buccal patch, strip, permeable pad or bag, etc.

For vaginal transmucosal delivery, the composition is formulated as a foam tampon, foam ring, foam pessary, foam suppository or foam sponge. Each of these may be conveniently incorporated into an intravaginal device, such as, for example, a conventional tampon, vaginal ring, pessary, suppository or vaginal sponge.

For labial transepithelial delivery, the foam or film will take on the structure conveniently attachable to labia, such as strip, pillow, pad, butterfly bandage, etc.

For scrotal transepithelial delivery, the composition is preferably formulated as a liquid or semi-liquid which is conveniently sprayed or otherwise applied to scrotum.

For transepithelial scrotal delivery, the foam or film is formulated as strip, attachable or sprayed on as a gellable film.

For a low release, bioadhesive foam tablets, strips, pads, or films consist essentially of hydroxypropyl cellulose and polyacrylic acid. These foams or films release drugs for up to five days once they are placed on or in close proximity of labial or scrotal epithelium.

For all these transmucosal administrations, the drug can also be first formulated as a solution, suspension, cream, lotion, paste, ointment or gels which can be incorporated into the foam or film and applied to the nasal or buccal cavity or vagina, labia or scrotum.

The choice of additional suitable additives and excipient depends on the exact nature of the particular transmucosal delivery route and the form in which the drug is delivered. Thus, the actual formulation depends on the properties of the pharmacological agent and on whether the active ingredient(s) is/are to be formulated into a foam or film or indirectly into a cream, lotion, foam, ointment, paste, solution, or gel, which is then incorporated into the foam or film, as well as on the identity of the active ingredient(s).

2. Devices

A therapeutic foam or film according to the invention can be a stand alone device or it may become a part of a more complex assembly comprising as one component the foam or film and as a second component a device or formulation made of a different material than foam or film described herein. Such other device may be in the form of, for example, a structural device such as a strip, pad, sphere, pillow, tampon, tampon-like device, vaginal ring, sponge or pessary, or it may be in a form of a formulation, such as a tablet, paste, suppository, bioadhesive tablet, bioadhesive microparticles, cream, lotion, ointment, or gel.

The structural device such as the tampon can be completely or partially coated or covered with the foam or film or the foam or film may be inserted inside of the device or into certain part of the device in any convenient arrangement.

In the alternative, the drug could be incorporated into the non-foam, non-film device and an empty foam or film composition could be used for coating or covering such device solely for the purpose of control of release rate.

IV. Routes of Delivery

The present invention concerns a polymer foam or film for delivery of therapeutic agents to and through nasal, oral or vaginal mucosal epithelium as well as through the cornified or noncornified epithelium of labia and scrotum. In particular, the invention concerns a solid, semi-solid or liquid polymeric foam or film having a therapeutic agent incorporated therein wherein said agent is released from said foam or film upon placement of said foam or film on the surface of nasal, buccal or vaginal mucosa, labia or scrotum. The foam of the invention has a controllable rate of gelling, swelling and degradation.

Treatment of various diseases, such as osteoporosis, inflammation, pain, prostate cancer and other neoplastic growths, fungal, bacterial, viral or parasitic infections and other medical conditions using a method of invention involves contacting the nasal, buccal, vaginal, labial or scrotal epithelium directly with a therapeutic agent suitable for treatment of such condition. Such direct contact permits an immediate, continuous and efficacious treatment of various diseases or medical conditions. Systemic drug delivery using transepithelial route eliminates inactivation of the agent by gastrointestinal tract or by liver metabolism. Such direct treatment also permits use of only such a dosage of the agent as is therapeutically required for treatment of the affected tissue.

For each of these treatments, the drug is formulated differently, as described. Briefly, the active drug is formulated to adhere to and directly cross or be transported through the mucosal, labial or scrotal epithelium. For transepithelial delivery to the general circulation, if necessary and appropriate for the properties of the drug, the additives which promote adhesion to transport and penetration of the drug through the nasal, buccal, vaginal, labial or scrotal epithelium are added.

A. Vaginal Delivery

The vaginal drug delivery system provides a sustained delivery of the drug to the vaginal epithelium for the treatment of various conditions including dysmenorrhea, osteoporosis, neoplastic growth, migraine, neurodegenerative diseases, vaginal or systemic infections, among others.

The vaginal delivery may be achieved by the foam device or film having a drug incorporated therein or it can be a solid object delivery system such as a conventional vaginal tampon, ring, pessary, tablet or suppository, for example, coated with or containing the foam or film. Alternatively, it can be a paste or gel incorporated into the foam or film having a sufficient thickness to maintain prolonged vaginal epithelium contact. Alternatively, the foam or film can provide a coating on a suppository wall or a sponge or other absorbent material impregnated with a liquid drug containing solution, lotion, or suspension of bioadhesive particles, for example. Any form of drug delivery system which will effectively deliver the treatment agent to the vaginal epithelium is intended to be included within the scope of this invention.

Intravaginal topical delivery comprises contacting the vaginal epithelium and mucosa with a foam or film composition comprising a therapeutically effective agent alone or in admixture with a carrier, mucoadhesive agent, sorption enhancer or penetration promoter.

Intravaginal delivery is achieved either directly by delivering the foam or film composition of the invention to-the vagina or by delivering the composition of the invention to the vagina incorporated into a vaginal device, as described above. The foam or film composition or the device, coated or incorporated therewith, is placed into a close contact with or into a close proximity of the vaginal epithelium wherein the agent is either released from the composition or device or released from the foam or film device and either directly or through the action of the mucoadhesive compound it comes into a contact with or adheres to the vaginal epithelium and mucosa where it penetrates the vaginal wall and is delivered to the uterus and/or to the blood circulation by being absorbed or transported through vaginal mucosa.

Delivery of the drugs through the vaginal mucosa using the current foams or films significantly improves systemic bioavailability and greatly increases concentrations of these drugs in the plasma.

B. Buccal Delivery

Transepithelial foam or film for buccal delivery of drugs permits transport of the drug into the systemic circulation directly through the nasal mucosa, thereby avoiding invasive intravenous or less effective oral administration.

In one embodiment, this invention concerns buccal delivery systems that are designed to interact with the epithelium lining the oral cavity wherein drug released from these devices may act topically on the buccal mucosa or successfully traverse the barrier of the buccal epithelium and reach mucosal and submucosal areas where they gain access to the systemic circulation for distribution to targets distinctly separated from the site of administration.

Drug delivery via the buccal route is applicable to patients of both genders, achieves high compliance since it is non-invasive and offers easy access to the site of administration. The buccal mucosa is rich in blood vessels facilitating access to systemic circulation. Furthermore, drug absorbed from the buccal mucosa will avoid hepatic first-pass metabolism similar to the vaginal route.

C. Nasal Delivery

In yet another embodiment, this invention also concerns administration of foam and film drug delivery devices to the nasal mucosa where incorporated drug may be released to the nasal epithelium or permeates the epithelial barrier to reach deeper mucosal tissue, where it may gain access to the systemic circulation for distribution. The nasal route has the advantage of providing rapid absorption with little or no degradation of drugs that have systemic targets since blood drainage from the nasal cavity also bypasses hepatic first-pass metabolism. This route is well-received by patients due to ease in administration of nasal preparations. Of particular interest are nasal delivery approaches for biotechnology-based drugs such as proteins that are designed to interact with the body immune system and boost immune defense (i.e., vaccines). Access to the immune system through the nose is provided only a few cell layers below the epithelium in form of the nasal-associated lymphatic tissue (NALT).

D. Labial Delivery

The current invention concerns delivery through the external non-cornified mucosal labial epithelium.

The foam or film the invention comprises administration of therapeutic and/or palliative anti-inflammatory, analgesic, chemotherapeutic, antineoplastic, antiosteoporotic, antifungal, antibacterial, antiviral or parasiticidal drugs to the non-cornified labial epithelium or through this barrier to deliver the pharmacologically active agents directly to the systemic circulation.

The foam or film composition or the medicated device is applied once, twice or several times a day, as needed, or according to a treatment regimen. The device, or its active part, such as for example a pad containing or covered with the foam or film composition, is typically provided in dry or wet form or may be wetted prior insertion.

The foam or film female device for drug delivery through labial epithelium is typically an insert, such as a tape, small pillow, minipad, small preferably rectangular pad or combination of two tapes or pads connected in butterfly-like fashion or one or two of these inserts attached to labia may be held in place with vaginal insert. The advantage of labial administration is that two devices and/or both sides of the device, be it the pad or the tape, can be medicated and two of these inserts may be applied at the same time along each side of clitoris.

One embodiment of the invention is a female foam or film device having a design of a labial butterfly pad, a pair of labial pads or a combination of a labial butterfly with a vaginal insert to hold the labial device in place. Both above devices are modified for containment of, or to accept, include or be impregnated with, a pharmacological agent formulated as a cream, lotion, foam, ointment, microparticles, nanoparticles, microemulsions, solution, or gel incorporated within said device.

Alternatively, the drug can be incorporated into a coating on a foam pad or sponge, or included within the foam pad as a suppository, sponge, tablet or other absorbent material may be impregnated with a liquid, drug containing solution, lotion, or suspension of bioadhesive particles, shaped into a pad may be used.

The female device for drug delivery through labia is generally any structure which can be attached or applied to labia. Device may be stand alone or attached to some structural support, such as a slip.

Typically, additionally to the devices described above, the female device may be a foam tape, adhesive tape, bandage, pad, pouch or bag which can be attached to the labia directly or is mounted into some structural support, such as a slip or strap, etc.

The device may optionally include a battery powered heating device to enhance blood flow and/or promote drug release and delivery. The battery is either attached to the pad or may be attached to the waistband of the slip or strap.

E. Scrotal Delivery

The foam or film of the invention permits administration of therapeutic anti-inflammatory, analgesic, chemotherapeutic, antiosteoporotic, antineoplastic, antifungal, antibacterial, antiviral or parasiticidal pharmacological agents to the cornified scrotal epithelium or through this barrier to deliver the pharmacologically active agent to prostate, testes or directly to the systemic circulation for systemic drug delivery.

The invention concerns a discovery that many of the problems noted with systemic delivery could be overcome by focusing the delivery of drug therapy directly to the non-mucosal scrotal epithelium using a topical composition or a device comprising a specially formulated therapeutical agent. The specially formulated foam or film composition promotes adhesion of the drug released from the device to the scrotum for transscrotal delivery. Optionally, such composition comprises additional components that enhance drug penetration and absorption through scrotal epithelium.

The method for transscrotal treatment encompasses a typical topical treatment comprising contacting the lightly cornified scrotal epithelium directly with the drug or with the device comprising the drug, for extended periods of time for as long as needed, by providing a topical foam or film composition or a device comprising a topical composition comprising the drug formulated in combination with at least a mucoadhesive agent to promote adherence of the drug to the scrotal epithelium and, optionally, with penetration enhancer.

One embodiment of the invention concerns a male device made of or coated with foam or film for delivery of a pharmacological agent through non-mucosal lightly cornified scrotal tissue. The device provides a continuous contact with the scrotal epithelium thereby asserting a therapeutic effect of the composition of the invention incorporated therein.

Typically, the male device is a foam or film tape, adhesive tape, bandage, pad, or set of tapes, bandages or pads, pouch or bag which can be attached to the scrotum directly or is mounted into some structural support, such as a strap, athletic supporter, suspender, etc., but it may also be a foam or film gel sprayed on the scrotum.

The foam or film compositions or the foam or film coated devices are administered or applied to the nasal, oral or vaginal cavity or to labia or scrotum once, twice or several times a day, as needed, or according to a treatment regimen. It may be applied once and left on the covering epithelium for several hours or days or it may be applied repeatedly in various intervals. The device, or its active part, such as for example a stand-alone foam or film coated pad or pad containing the composition is typically provided in dry or wet form or may be wetted prior to emplacement into the nasal, oral or vaginal cavity or labia or scrotum.

### EXAMPLE 1

### Ketoconazole Foam

This example illustrates preparation of the foam containing ketoconazole.

Polyethylene glycol 400 was obtained from Fluka Chemika, alginic acid sodium salt was obtained from Sigma-Aldrich, and ketoconazole (USP 24, micronized) was obtained from Quimica Sintetica S.A.

Ketoconazole was dissolved in polyethylene glycol (PEG) 400 to form a homogeneous 10 mg/mL solution. Alginic acid sodium salt was dissolved in distilled water to produce a 5.0 w/w % solution. Forty-five milliliters (45.0 mL) of the alginic acid solution was combined with 5.0 mL of the ketoconazole/PEG 400 solution, and these solutions were mixed together at 70° C. for 15 minutes. Five milliliter (5.0 mL) aliquots of this solution were poured into 5.0 mL plastic syringes and frozen at −80° C. Frozen cylindrical samples were subsequently removed from the syringe molds and lyophilized using a Virtis Unitop 1000L shelf lyophilizer. Cylindrical ketoconazole-containing polymeric foams resulted.

### EXAMPLE 2

### Preparation of Drug-Containing Foam for Vaginal Delivery

This example describes a process for preparation of a foam for topical vaginal delivery of ketoconazole.

Ketoconazole (USP 24, micronized) was obtained from Quimica Sintetica S.A. Hydroxypropyl methylcellulose (Methocel@ K, HPMC K15M), was obtained from Dow Chemical, Midland, Michigan. Polysorbate 80 (Tween@ 80) was obtained from Spectrum Chemical Manufacturing Corp., Gardena, Calif.

Foams were prepared by adding 1.0 gm of Tween 80 to 100.0 mL of distilled water in a beaker. The solution was heated to 80° C. and 2.5 gm of Methocel were subsequently added. Mechanical stirring was used to prepare a homogenous solution. The solution was cooled to 60° C. and 2.0 gm ketoconazole was added. Mechanical stirring was used to completely mix the resulting formulation.

Eighteen 5.0 mL plastic syringes were filled with the drug-containing solution and placed into a freezer at −80° C. for one hour. Frozen cylinders of the solution were then expelled from the syringes and placed in a Virtis Unitop 1000L lyophilizer. The cylinders were subsequently lyophilized to produce cylindrical ketoconazole-containing foam samples.

### EXAMPLE 3

### Preparation of Drug-Containing Foam for Topical Vaginal Delivery

This example describes a process for preparation of a foam for transvaginal delivery of ketoconazole.

Ketoconazole (USP 24, micronized) was obtained from Quimica Sintetica S.A. Hydroxypropyl methylcellulose (Methocel@ K, HPMC K15M) was obtained from Dow Chemical, Midland, Mich. Polysorbate 80 (Tween@ 80) was obtained from Spectrum Chemical Manufacturing Corporation, Gardena, Calif. All other chemicals were obtained from Sigma Aldrich, St. Louis, Mo.

A citric acid/phosphate buffer solution (pH=5.0) was prepared using a 0.1 molar citric acid solution and a 0.2 molar disodium phosphate solution. One hundred milliliters of the solution was prepared by adding 49.0 mL of the citric acid solution to 51.0 mL of the disodium phosphate solution.

Foams were prepared by adding 1.0 gm of Tween to 80 to 100.0 mL of the citric acid/phosphate buffer solution in a beaker. The solution was heated to 80° C. and 2.5 gm of Methocel were subsequently added. Mechanical stirring was used to prepare a homogenous solution. The solution was cooled to 60° C. and 2.000 mg ketoconazole was added. Mechanical stirring was used to completely mix the resulting formulation.

Eighteen 5.0 mL plastic syringes were filled with the drug-containing solution and placed into a freezer at −80° C. for one hour. Frozen cylinders of the solution were then expelled from the syringes and placed in a Virtis Unitop 1000L lyophilizer. The cylinders were subsequently lyophilized to produce cylindrical ketoconazole-containing foam samples.

### EXAMPLE 4

### Preparation of Drug-Containing Foam for Transvaginal Delivery

This example describes a process for preparation of a foam for transvaginal delivery of ketoconazole.

Foams were prepared by adding 2.5 gm of Methocel to 100.0 mL of distilled water and heating the solution to 80° C. Mechanical stirring was used to prepare a homogenous solution. The solution was cooled to 60° C. and 2.0 gm ketoconazole was added.

Eighteen 5.0 mL plastic syringes were filled with the drug-containing solution and placed into a freezer at −80° C. for one hour. Frozen cylinders of the solution were then expelled from the syringes and placed in a Virtis Unitop 1000L lyophilizer. The cylinders were subsequently lyophilized to produce cylindrical ketoconazole-containing foam samples.

### EXAMPLE 5

### Ketorolac Containing Foam

This example describes preparation of the ketorolac containing foam using alginic acid/hydroxypropyl methylcellulose substrates.

A solution was prepared by mixing 0.2015 g ketorolac tromethamine with 100.0 ml deionized water at 70-80 C. with stirring, followed by adding 1.2507 g hydroxypropyl methylcellulose followed by 1.2503 g alginic acid with continued stirring. The warm solutions were dispensed into 10 ml plastic syringes in 10 ml aliquots. The samples were frozen at −80° C. for 18 hr. After brief warming at room temperature, the samples were ejected from the syringes onto a metal pan precooled to −40° C. The samples were converted to foams by freeze-drying under vacuum at −20° C. for 117 hr, followed by warming to ambient temperature for 5 hr while under vacuum. The resulting foams were stored under dry conditions.

### EXAMPLE 6

### Alginic Acid Foam Containing Ketorolac

This example describes preparation of alginic acid foam containing ketorolac tromethamine.

A solution was prepared by mixing 0.2002 g ketorolac tromethamine with 100.0 ml deionized water at 70-80° C. with stirring, followed by adding 2.5023 g alginic acid with continued stirring.

The warm solutions were dispensed into 10 ml plastic syringes in 10 ml aliquots. The samples were frozen at −80° C. for 18 hr. After brief warming at room temperature, the samples were ejected from the syringes onto a metal pan precooled to −40° C. The samples were converted to foams by freeze-drying under vacuum at −20° C. for 117 hr, followed by warming to ambient temperature for 5 hr while under vacuum. The resulting foams were stored under dry conditions.

### EXAMPLE 7

### Alginic Acid Film Containing Ketorolac

This example describes preparation of alginic acid film containing ketorolac tromethamine.

A solution was prepared by mixing 2.5 g alginic acid with 50.0 ml deionized water at 80° C. with stirring. After cooling to room temperature, 100 mg of ketorolac was added and stirred for 1 hr. The solution was poured into 4-inch diameter molds and was allowed to dry at room temperature for 70 hr. The resulting films were stored under dry conditions.

### EXAMPLE 8

### Hydroxypropyl Methylcellulose-Avicel Foam

This example describes preparation of foam using hydroxypropyl methylcellulose and microcrystalline cellulose derivative as a substrate.

A solution was prepared by mixing 1.0046 hydroxypropylmethyl cellulose and 20.0192 g avicel PH-101 microcrystalline cellulose with 79.0 g deionized water at about 70° C. with stirring. The warm solution was dispensed into 5 ml plastic syringes in 5 ml aliquots. After cooling to room temperature, the samples were frozen at −80° C. for 2 hr. After brief warming at room temperature, the samples were ejected from the syringes onto a metal pan precooled to −20° C. The samples were converted to foams by freeze-drying at −20° C. for 90 hr and −10° C. for 2 hr. The samples were then warmed to ambient temperature under vacuum for 22 hr. The resulting foam rods were stored under dry conditions.

### EXAMPLE 9

### Hydroxypropyl Methylcellulose Foam

This example describes preparation of foam using hydroxypropyl methylcellulose and microcrystalline cellulose derivative as a substrate.

A solution was prepared by mixing 5.0014 Hydroxypropylmethyl Cellulose and 5.0050 g Avicel PH-101 microcrystalline cellulose with 90.0 g deionized water at about 70° C. with stirring. The warm solution was dispensed into 5 ml plastic syringes in 5 ml aliquots. After cooling to room temperature, the samples were frozen at −80° C. for 2 hr. After brief warming at room temperature, the samples were ejected from the syringes onto a metal pan precooled to −20° C. The samples were converted to foams by freeze-drying at −20° C. for 90 hr and −10° C. for 2 hr. The samples were then warmed to ambient temperature under vacuum for 22 hr. The resulting foam rods were stored under dry conditions.

### EXAMPLE 10

### Hydroxvpropyl Methylcellulose Foam

This example describes preparation of foam using hydroxypropyl methylcellulose and microcrystalline cellulose derivative as a substrate.

A solution was prepared by mixing 5.0044 hydroxypropylmethyl cellulose and 20.0017 g avicel PH-101 microcrystalline cellulose with 75.0 g deionized water at about 70° C. with stirring. The warm solution was dispensed into 5 ml plastic syringes in 5 ml aliquots. After cooling to room temperature, the samples were frozen at −80° C. for 2 hr. After brief warming at room temperature, the samples were ejected from the syringes onto a metal pan precooled to −20° C. The samples were converted to foams by freeze-drying at −20° C. for 90 hr and −10° C. for 2 hr. The samples were then warmed to ambient temperature under vacuum for 22 hr. The resulting foam rods were stored under dry conditions.

### EXAMPLE 11

### Alginic Acid-HPMC Foams Containing Transcutol and Ketorolac Tromethamine

This example describes preparation of alginic acid/HPMC foams containing penetration enhancer transcutol and ketorolac tromethamine.

A solution was prepared by mixing 0.20 g ketorolac tromethamine with 100.0 ml deionized water at 70-80° C. with stirring, followed by adding 1.25 g hydroxypropyl methylcellulose followed by 1.25 g Alginic Acid with continued stirring. The warm solutions were dispensed into 10 ml plastic syringes in 10 ml aliquots. The samples were frozen at −80° C. for 18 hr. After brief warming at room temperature, the samples were ejected from the syringes onto a metal pan precooled to −40° C. The samples were converted to foams by freeze-drying under vacuum at 20° C. for 117 hr, followed by warming to ambient temperature for 5 hr while under vacuum. Foam rods, cut to about 4 cm length and weighing about 160 mg, were sprayed with about 1.0 ml of 1.6% transcutol tromethamine in methylene chloride. The methylene chloride was evaporated using gentle heat, leaving about 16 mg of transcutol tromethamine in the foam rod. The resulting foams were stored under dry conditions.

### EXAMPLE 12

HPMC Foams Containing Cyclodextrin B
This example describes preparation of HPMC foams
containing Cyclodextrin B.

| Composition: | Foam #1 | Foam #2 | Foam #3 |
|---|---|---|---|
| HPMC | 2.4992 g | 2.5100 g | 2.4906 g |
| | (95.21%) | (91.0%) | (83.2%) |
| Beta-Cyclodextrin | 0.1258 g | 0.2940 g | 0.5015 g |
| | (4.79%) | (9.02%) | (16.8%) |
| Water | 97.5 g | 97.5 g | |
| BCD:HPMC Ration | 1:20 | 1:10 | 1:5 |

Solutions were prepared by mixing hydroxypropylmethyl cellulose, β-gyclodextrin, and deionized water at about 70° C. with stirring. The warm solution was dispensed into 5 ml plastic syringes in 5 ml aliquots. After cooling to room temperature, the samples were frozen at −80° C. for 35 min. After brief warming at room temperature, the samples were ejected from the syringes onto a metal pan precooled to −20° C. The samples were converted to foams by freeze drying at −20° C. for 17 hr and −10° C. for 49 hr. The samples were then warmed to ambient temperature under vacuum for 4.5 hr. Soft white foam rods were produced in all cases. The foam rods were stored under dry conditions.

### EXAMPLE 13

### Alginic Acid Film

This example describes preparation of alginic acid film.

Alginic acid sodium salt was obtained from Sigma-Aldrich and dissolved in distilled water to produce a 5.0 w/w % solution. The alginic acid and water were mixed for at least 2 hours at 80° C. using a magnetic stir bar to form a homogeneous solution. Layers of this viscous alginic acid solution, with thicknesses ranging from 300 mm to 2.0 mm, were coated onto glass plates. (20×20 cm²) using a manual thin layer chromatography (TLC) plate coater (CAMAG, Switzerland). The layers of solution were allowed to dry for 24 hours at 25° C., and the resultant polymer films were removed from the glass plates. Clear, flexible, hydrophilic alginic acid films resulted.

### EXAMPLE 14

### Alginic Acid Alendronate Sodium Film

This example describes preparation of alginic acid film comprising alendronate.

Alginic acid sodium salt was obtained from Sigma-Aldrich and dissolved in distilled water to produce a 5.0 w/w % solution using the above described method. Alendronate sodium (Lot #ASFPGO04) was obtained from Albany Molecular Research, Albany, N.Y., and 50.6 mg was added to 25.0 mL of the alginic acid solution. The solution was agitated at 25° C. for at least one hour in a plastic 50 mL conical tube using a wrist action shaker to form a clear, homogeneous solution. The viscous alginic acid alendronate sodium solution was coated onto glass plates (20×20 cm²) in layers approximately 1.0 mm thick using a manual thin layer chromatography (TLC) plate coater (CAMAG, Switzerland). The layers of solution were allowed to dry for 24 hours at 25° C., and the resultant polymer films were removed from the glass plates. Clear, flexible, hydrophilic alginic acid alendronate sodium films resulted.

### EXAMPLE 15

### Alginic Acid Metoclopramide Hydrochloride Film

This example describes preparation of alginic acid film comprising metoclopramide.

Alginic acid sodium salt was obtained from Sigma-Aldrich and dissolved in distilled water to produce a 5.0 w/w % solution using the above described method. Metoclopramide hydrochloride was obtained from ICN Biomedicals, Inc., Aurora, Ohio, and 51.6 mg was added to 25.0 mL of the alginic acid solution. The solution was agitated at 25° C. for at least one hour in a plastic 50 mL conical tube using a wrist action shaker to form a clear, homogeneous solution. The viscous alginic acid metoclopramide hydrochloride solution was coated onto glass plates (20×20 cm²) in layers approximately 1.0 mm thick using a manual thin layer chromatography (TLC) plate coater (CAMAG, Switzerland). The layers of solution were allowed to dry for 24 hours at 25° C., and the resultant polymer films were removed from the glass plates. Clear, flexible, hydrophitic alginic acid/metoclopramide hydrochloride films resulted.

### EXAMPLE 16

### HPMC/Alendronate Sodium Film

This example describes procedure used for preparation of alendronate containing film.

Hydroxypropyl methylcellulose (HPMC) was obtained from The Dow Chemical Company (Methocel K15M) and dissolved in distilled water to produce a 2.5 w/w % solution using the above described method. Alendronate sodium (Lot #ASFPGO04) was obtained from Albany Molecular Research, Albany, N.Y., and 49.0 mg was added to 25.0 mL of the HPMC solution. The solution was agitated at 25° C. for at least one hour in a plastic 50 mL conical tube using a wrist action shaker to form a clear, homogeneous solution. The viscous HPMC/alendronate sodium solution was coated onto glass plates (20×20 cm²) in layers approximately 1.0 mm thick using a manual thin layer chromatography (TLC) plate coater (CAMAG, Switzerland). The layers of solution were allowed to dry for 24 hours at 25° C., and the resultant polymer films were removed from the glass plates. Clear, flexible, hydrophilic HPMC/alendronate sodium films resulted.

### EXAMPLE 17

### HPMC/Metoclopramide Hydrochloride Film

This example describes procedure used for preparation of film containing metoclopramide.

Hydroxypropyl methylcellulose (HPMC) was obtained from The Dow Chemical Company (Methocel K15M) and dissolved in distilled water to produce a 2.5 w/w % solution using the above described method. Metoclopramide hydrochloride was obtained from ICN Biomedicals, Inc., Aurora, Ohio, and 50.8 mg was added to 25.0 mL of the HPMC solution. The solution was agitated at 25° C. for at least one hour in a plastic 50 mL conical tube using a wrist action shaker to form a clear, homogeneous solution. The viscous HPMC/metoclopramide hydrochloride solution was coated onto glass plates (20×20 cm²) in layers approximately 1.0 mm thick using a manual thin layer chromatography (TLC) plate coater (CAMAG, Switzerland). The layers of solution were allowed to dry for 24 hours at 25° C., and the resultant polymer films were removed from the glass plates. Clear, flexible, hydrophilic HPMC/metoclopramide hydrochloride films resulted.

### EXAMPLE 18

### Preparation of Foams or Films Containing Pharmacological Agent

This example describes the preparation of foams or films for mucosal, transmucosal, scrotal, transscrotal, labial or tarns-labial delivery of various pharmacological agents.

A foam or film prepared according to any of the Examples 1 through 17 for mucosal, transmucosal, labial, translabial, scrotal or transscrotal administration of each one of the following drugs at the indicated dose: aspirin (975 mg), piroxicam (20 mg), indomethacin (50 mg), fenamate (500 mg), sulindac (200 mg), nabumetone (750 mg), detorolac (10 mg), ibuprofen (200 mg), phenylbutazone (50 mg), bromfenac (50 mg), naproxen (550 mg), lidocaine (100 mg), mepivacaine (0.2 mg), etidocaine (200 mg), bupivacaine (100 mg), 2-chloroprocaine hydrochloride (100 mg), procaine (200 mg), tetracaine hydrochloride (20 mg), diltiazem (60 mg), israpidine (10 mg), nimodipine (30 mg), felodipine (450 mg), nifedipine (90 mg), nicardipine (30 mg), ritodrine (150 mg), bepridil (300 mg), dofetilide (1 mg), almokalant (1 mg), sematilide (1 mg), ambasilide (1 mg), azimilide (1 mg), tedisamil (100 mg), sotalol (240 mg), ibutilide (1 mg), terbutaline (5 mg), salbutamol (1 mg), piroxicam (20 mg), metaproterenol sulphate (20 mg), nitroglycerin (3 mg), isosorbide dinitrate (40 mg), isosorbide mononitrate (120 mg). Other drugs, in amounts as described above in Section D, may be formulated in the same fashion.

The quantity of the drug dosage needed to deliver the desired dose depends on the concentration of the active ingredient in the composition and the amount of the penetration enhancer or mucoadhesive agent. The therapeutic dosage range for vaginal transmucosal administration of the compositions of the present invention will vary with the size of the patient.

### EXAMPLE 19

### Preparation of Film Solution Containing Ketorolac for Transmucosal Nasal Delivery

This example describes the preparation of a transmucosal ethoxydiglycol-containing nasal composition.

Using a high-shear mixer, 1 g ketorolac tromethamine, 1.5 g Tween 80, 1.0 g polycarbophil, 0.05 g sodium chloride, and 2.5 g sorbitol were dispersed in 44 g deionized water. The solution is sterilized by passing it through a 0.2 micron Millipore filter. The resulting translucent mixture was suitable for spraying or spreading onto nasal tissue.

### EXAMPLE 20

### Preparation of a Transmucosal Foam Gel Composition Containing Ketorolac

This example describes the preparation of a transmucosal gel composition containing ketorolac for transvaginal delivery.

Ketorolac tromethamine (1 g), Tween 80 (5 g), propylene glycol (10 g), and ethoxydiglycol (Transcutol P) (15 g) were added to deionized water (44 g) heated to 70-80° C. in a 200 ml beaker while mixing with a high-shear mixer. Triacetin (20 g) and hydroxypropyl methylcellulose (5 g) were added gradually while maintaining the temperature and mixing. Upon cooling, the viscosity increased until the mixture had the consistency of a gel.

### EXAMPLE 21

### Preparation of Pamidronate Containing Buccal Foam Pad

This example describes preparation of pamidronate containing buccal pad.

The dose of unlabeled pamidronate, commercially available from Sigma, St. Louis, Mo, was 0.2 mg/kg body weight. The pamidronate buccal pad is prepared by soaking the cotton, hydroxypropyl methyl cellulose or foam pad in the solution of pamidronate prepared similarly as described in Example 4.

### EXAMPLE 22

### Mucoadhesive Buccal Film

This example describes the preparation of a mucoadhesive buccal film containing the peptide drug salmon calcitonin as the hydrophilic drug for transmucosal delivery.

Salmon calcitonin (MW=3.4 kD) was purchased from Bachem (Torrance, Calif.). 50:50 Poly(D,L-lactide-co-glycolide) was obtained from Boehringer Ingelheim (Ingelheim, Germany). Chitosan glutamate salt, medical grade (MW=150 kD) was received from Pronova Biochemical AS (Oslo, Norway). Methanol, dichlormethane, and glycerol were purchased from Sigma Chemical (St Louis, Mo.). An oil-in-water emulsion was formed by dropping 5 g of a solution prepared with 0.5 mL of 2% (w/w) salmon calcitonin in methanol and 4.5 mL of 20% (w/w) poly(D,L-lactide-co-glycolide in chloroform into a chitosan aqueous solution (2%, w/w) with 0.5% (w/w) glycerol under stirring (9500 rpm) at 15° C. The mixture was maintained under stirring for 20 minutes, spread as a thin layer onto a glass plate using a CAMAG TLC plate coater, and kept at 30° C. to allow solvent evaporation.

What is claimed is:

1. A polymer foam or film composition for delivery of pharmacologically effective agents topically to nasal, buccal, vaginal, labial or scrotal epithelium or through nasal, buccal, vaginal, labial or scrotal epithelium into a systemic circulation, said composition comprising at least one substrate polymer or a mixture of substrate polymers and a pharmacologically effective agent.

2. The composition of claim 1 wherein said substrate polymer is hydrophilic, hydrophobic or a mixture of both.

3. The composition of claim 3 wherein said substrate polymer is selected for the group consisting of hydropropyl methylcellulose, gelatin, alginic acid, alginic acid sodium salt, polyethyleneglycol, pectin, collagen, poloxamer, carbopol, microcrystalline cellulose, polyacrylic acid, polyethylene glycol, polypropylene glycol, divinyl glycol, polyethylene oxide, polypropylene oxide, carboxymethyl cellulose, hydroxypropylmethyl cellulose, hydroxyethyl cellulose, polylactide, polyglycolide, polymethacrylic acid, poly-γ-benzyl-L-glutamate, polypropylene fumarate, poly-ε-caprolactone, poly-butylene terephthalate, polyvinyl alcohol, polyvinyl ether, poly-1-vinyl-2-pyrrolidinone, 2,5-dimethyl-1,5-hexadiene, divinyl benzene, polystyrene-divinyl benzene, polybisp-carboxy-phenoxypropane-co-sebacic acid, poly-β-hydroxybutyrate, poly-β-butyrolactone, tetraethylorthosilicate and dimethyldiethoxysilane.

4. The composition of claim 2 wherein the polymer is hydropropyl methylcellulose, gelatin, alginic acid, alginic acid sodium salt, polyethyleneglycol, pectin, collagen, poloxamer, carbopol or microcrystalline cellulose.

5. The composition of claim 4 further comprising a penetration enhancer, sorption promoter, mucoadhesive agent, hydrophilic or hydropholic release modifier, or a mixture thereof.

6. The composition of claim 5 wherein said mucoadhesive agent is selected form the consisting of hydroxypropyl methylcellulose, carboxymethylcellulose, polylactide-coglycolide, chitosan, chitosan ester or trimethylene chloride chitosan, sodium alginate, poloxamer, carbopol, pectin, polyacrylic acid, hyaluronic acid, polyvinyl alcohol, polyvinyl pyrrolidone, polycarbophil and carbopol,
wherein said penetration enhancer is selected from the group consisting of sodium caproate, sodium caprylate, sodium caprate, sodium laurate, sodium myristate, sodium palmitate, sodium palmitoleate, sodium oleate, sodium ricinoleate, sodium linoleate, sodium stearate, sodium lauryl sulfate, sodium tetradecyl sulfate, sodium laryl sarcosine, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodexoycholate, sodium glycol chenodesoxycholate, sodium cholylsarcosine, sodium N-methyl taurocholate, sodium tauro-24,25-dihydrofusidate, disodium polyoxyethylene-10 oleyl ether phosphate, esterification product of fatty alcohols, fatty alcohol ethoxylate with phosphoric acid or anhydride, ether carboxylate, succinylated monoglyceride, sodium stearyl fumarate, steaoryl propylene glycol hydrogen succinate, mono/diacetylated tartaric acid ester of mono- and diglycerides, citric acid esters of mono- and diglycerides, glyceryl-lacto esters of fatty acids, lactylic ester of fatty acids, alginate salt, ethoxylated alkyl sulfate, alkyl benzene sulfone, α-olefin sulfonate, acyl isethionate, acyl taurate, alkyl glyceryl ether sulfonate, octyl sulfosuccinate disodium, disodium undecylenamideo-MEA-sulfosuccinate, phosphatidic acid, phosphatidyl glycerol, polyacrylic acid, hyaluronate sodium, glycyrrhetinic acid, ethylene diamine tetraacetate, sodium citrate, chitosan, trimethyl chitosan, poly-L-arginine chitosan, poly-L-lysine chitosan, aminated gelatin, hexadecyl triammonium chloride, decyl trimethylammonium chloride, cetyl trimethylammonium chloride, alkyl benzyldimethylammonium chloride, diisobutyl phenoxyethoxydimethyl benzylammonium chloride, ethyl pyridinium chloride, isopropyl pyridinium chloride, N-lauryl,N,N-dimethylglycine, N-capryl,N,N-diethylglycine, polyoxyethylene-coconut amine, poly-L-lysine, poly-L-arginine, lecithin, lysolecithin, hydroxylated lecithin, lysophosphatidylcholine, phosphatidylcholine, phosphatidylethanolamine, phosphatidylserine, didecanoyl-L-α-phosphatidylcholine, laurolycarnitine, acylcarnitine, palmitoyl-D,L-carnitine, polyoxyethylene lauryl ether, polyoxyethylene monooleyl ether, ethoxydiglycol, polyoxyethylene nonylphenol polyoxyethylene octylphenol ether, polyoxyethylene cholesterol ether, polyoxyethylene soya sterol ether, α-cyclodextrin, β-cyclodextrin, γ-cyclodextrin, dimethyl-β-cyclodextrin, methylated-β-cyclodextrin, 2-hydroxypropyl-β-cyclodextrin, sorbitol, polyoxyethylene glycol ester, polyoxyethylene glycerol fatty acid ester, polyoxyethylene glycerol fatty acid ester, polyoxyethylene glyceride, polyoxyethylene vegetable or hydrogenated oil, polyoxyethylene monooleate, polyoxyethylene dilaurate, polyoxyethylene mono and dioleate, polyoxyethylene glyceryl laurate, polyoxyethylene glyceryl oleate, propylene glycol oleate, propylene glycol stearate, polyoxyethylene sorbitan monooleate, polyoxyethylene tristearate, polyoxyethylene hydrogenated castor oil, polyoxyethylene almond oil, polyoxyethylene apricot kernel oil, polyoxyethylene caprylic glyceride, polyoxyethylene capric glyceride, lauroyl macrogol glyceride, and
wherein said release modifier is selected from the group consisting of polyethylene glycol 200, polyethylene glycol 8000, poloxamer, polyoxyethylene glycerylcocoate, carbopol, suppocire AS2X, suppocire CM, Witepsol H15, Witepsol W25, mineral oil, corn oil, paraffin oil, canola oil, castor oil, cottonseed oil, lecithin, peanut oil, sesame oil, soybean oil and hydrogenated vegetable oil.

7. The composition of claim 6 wherein said mucoadhesive agent is present in from about 0.5% to about 10% by weight, wherein said penetration enhancer is present in amount from about 0.1% to about 60% by weight, wherein said release modifier is present in amount from about to about 5% to about 70% by weight.

8. The composition of claim 7 further comprising pharmacologically acceptable additives or excipients.

9. The composition of claim 8 wherein said additives or excipients are solubilizing agents, buffering agents, fillers, preservatives, plasticizers, surfactants or anti-oxidants.

10. The composition of claim 9 wherein the substrate polymer, alone or in combination, is further combined with a pharmacologically effective agent selected from the group consisting of an anti-osteoporotic, non-steroidal anti-inflammatory, calcium channel antagonist, local anesthetic, potassium channel antagonists, β-adrenergic agonist, vasodilator, cyclooxygenase inhibitor, anti-fungal, antiviral, antimicrobial, antiparasitic, anti-epileptic, anti-migraine, anti-HIV, anti-neurodegenerative, anti-psychotic, chemotherapeutic or anti-neoplastic and opioid analgesic agent.

11. The composition of claim 10 wherein said nonsteroidal anti-inflammatory drug is selected from the group consisting of aspirin, ibuprofen, indomethacin, phenylbutazone, bromfenac, fenamate, sulindac, nabumetone, ketorolac, and naproxen;
wherein said calcium channel antagonist is selected from the group consisting of diltaizem, israpidine, nimodipine, felodipine, verapamil, nifedipine, nicardipine, and bepridil;
wherein said potassium channel blocker is selected from the group consisting of dofetilide, almokalant, sematilide ambasilide, azimilide, tedisamil, sotalol, piroxicam and ibutilide;
wherein said β-adrenergic agonist is selected from the group consisting of terbutaline, salbutamol, metaproterenol, ritodrine;
wherein said COX-2 or COX-1 inhibitor is selected from the group consisting of naproxen, ketoprofen, ketorolac, indomethacin, diclofenac, teroxicam, celecoxib, meloxicam and flosulide;
wherein said vasodilator is selected from the group consisting of nitroglycerin, isosorbide dinitrate, and isosorbide mononitrate;
wherein said bisphosphonate is selected from the group consisting of alendronate, clodronate, etidronate, pamidronate, tiludronate, ibandronate, zoledronate, alpadronate, residronate and neridronate;
wherein said antifungal agent selected from the group consisting of miconazole, terconazole, isoconazole, fenticonazole, tioconazole, fluconazole, nystatin, ketoconazole, clotrimazole, butoconazole, econazole, metronidazole and itraconazole;
wherein said antibacterial agent is selected from the group consisting of metronidazole, clindamycin, tetramycin, erythromycin, doxicycline, lumefloxacin, norfloxacin, afloxam, ciproflaxin, azitromycin, cefltoxime and doxicycline;
wherein said selected parasiticidal agent is metronidazole and clotrimazole;
wherein said antiviral agent is acyclovir or AZT;
wherein said anti-migraine agent is almotriptan, eletriptan, flovatriptan, naratriptan, rizatriptan, sumatriptan, zolmitriptan,. ergotamine, dihydroergotamine, bosentan and lanepitant;
wherein said anti-cancer agent is vincristine, cisplastin, doxorubicin, daunorubicin, etoposide, topotecan, irinotecan, paclitaxel, docetaxel, cyclophosphamide, methotrexate, and gemcitabine;
wherein said anti-HIV agent is saquinavir, ritonavir, indinavir, amprenavir, nelfinavir, lopinavir and ganciclovir; and
wherein said biotechnology-derived protein or peptide is insulin, calcitonin, vasopressin, luprolide, somatostatin, oxytocin, bivalirudin, integrilin, natrecor, abarelix, gastrine G17, peptide, ziconotide, cereport, interleukin, humanized antibodies and growth hormone.

12. The composition of claim 11 administered to a surface of a nasal, buccal, vaginal, labial or scrotal device.

13. The composition of claim 12 formulated as a foam.

14. The composition of claim 13 wherein the foam has a variable shape and size.

15. The composition of claim 14 wherein the foam is preformed into a device shaped as a sheet, tube, tampon, cylinder, pillow, strip, pad, sphere, tablet, ring, or bead.

16. The composition of claim 12 formulated as a film.

17. The composition of claim 16 wherein the foam has a variable thickness and size.

18. The composition of claim 12 wherein the film is used as a coating for a nasal, buccal, vaginal or labial device.

19. The composition of claim 18 wherein said foam or film is prepared by lyophilization or by aeration.

20. A device comprising a polymer foam or film composition of claims 1-18, said device suitable for delivery of therapeutically effective agents topically to a nasal, buccal, vaginal or labial cavity wherein said device is either coated with said composition or said composition is incorporated into said device.

21. The device of claim 19 wherein the device is a tampon, tampon-like device, ring, sponge, pessary, suppository, pillow, pad, strip, cylinder, sphere or bead and wherein the composition is a foam or film coating or a foam or film incorporated into said device.

22. A method for topical or systemic delivery of drugs to or through nasal, buccal, vaginal, labial or scrotal epithelium said method comprising a step of contacting the vaginal, nasal, buccal, labial or scrotal epithelium with a foam or film composition consisting essentially of a substrate polymer and a pharmacologically effective agent.

23. The method of claim 22 wherein pharmacologically effective agent is selected from the group consisting of an nonsteroidal anti-inflammatory, anti-prostaglandin, prostaglandin inhibitor, cyclooxygenase inhibitor, calcium channel blocker, potassium channel blockers, β-adrenergic agonists, vasodilator, antibiotic, antimycotic, bisphosphonate, anti-nausea, anti-psychotic, anti-migraine, anti-HIV, anti-cancer, chemotherapeutic a biotechnology derived protein or peptide, anti-epileptic, opioid analgesic,
wherein the amount of said pharmacological agent in the said composition administered to the mucosa is sufficient to deliver a therapeutically effective dose from about 0.01 to about 2000 mg of the pharmacological agent to the systemic circulation.

24. The method of claim 23 wherein said nonsteroidal anti-inflammatory drug is selected from the group consisting of aspirin, ibuprofen, indomethacin, phenylbutazone, bromfenac, fenamate, sulindac, nabumetone, ketorolac, and naproxen;
wherein said calcium channel antagonist is selected from the group consisting of diltaizem, israpidine, nimodipine, felodipine, verapamil, nifedipine, nicardipine, and bepridil;
wherein said potassium channel blocker is selected from the group consisting of dofetilide, almokalant, sematilide ambasilide, azimilide, tedisamil, sotalol, piroxicam and ibutilide;
wherein said β-adrenergic agonist is selected from the group consisting of terbutaline, salbutamol, metaproterenol, ritodrine;
wherein said cyclooxygenase inhibitor is selected from the group consisting of naproxen, ketoprofen, ketorolac, *indomethacin, diclofenac, teroxicam, celecoxib, meloxidam and flosulide;
wherein said vasodilator is selected from the group consisting of nitroglycerin, isosorbide dinitrate, and isosorbide mononitrate;
wherein said bisphosphonate is selected from the group consisting of alendronate, clodronate, etidronate, pamidronate, tiludronate, ibandronate, zoledronate, alpadronate, residronate and neridronate;
wherein said antifungal agent selected from the group consisting of miconazole, terconazole, isoconazole, fenticonazole, tioconazole, fluconazole, nystatin, ketoconazole, clotrimazole, butoconazole, econazole, metronidazole and itraconazole;
wherein said antibacterial agent is selected from the group consisting of metronidazole, clindamycin, tetramycin, erythromycin, doxicycline, lumefloxacin, norfloxacin, afloxam, ciproflaxin, azitromycin, cefltoxime and doxicycline;
wherein said selected parasiticidal agent is metronidazole and clotrimazole;
wherein said antiviral agent is acyclovir or AZT;
wherein said anti-migraine agent is almotriptan, eletriptan, flovatriptan, naratriptan, rizatriptan, sumatriptan, zolmitriptan, ergotamine, dihydroergotamine, bosentan and lanepitant;
wherein said anti-cancer agent is vincristine, cisplastin, doxorubicin, daunorubicin, etoposide, topotecan, irinotecan, paclitaxel, docetaxel, cyclophosphamide, methotrexate, and gemcitabine;
wherein said anti-HIV agent is saquinavir, ritonavir, indinavir, amprenavir, nelfinavir, lopinavir and ganciclovir; and
wherein said biotechnology-derived protein or peptide is insulin, calcitonin, vasopressin, luprolide, somatostatin, oxytocin, bivalirudin, integrilin, natrecor, abarelix, gastrine G17, peptide, ziconotide, cereport, interleukin, humanized antibodies and growth hormone.

25. The method of claim 24 wherein said composition is delivered through vaginal epithelium.

26. The method of claim 24 wherein said composition is delivered through nasal mucosa.

27. The method of claim 24 wherein said composition is delivered through buccal mucosa.

28. The method of claim 24 wherein said composition is delivered through scrotal epithelium.
